(19) United States
(12) Patent Application Publication
(10) Pub. No.: US 2025/0134500 A1
(43) Pub. Date: May 1, 2025

(54) Bone Harvester and Method for Processing a Bone

(71) Applicant: Incept, LLC, Lexington, MA (US)

(72) Inventors: Chun-Jen Liao, Zhubei City (TW); Yung-Chih Wu, Taoyuan City (TW); Pei Hsin Hsu, Taichung City (TW); Zhi Yu Chen, New Taipei City (TW); Amarpreet S. Sawhney, Lexington, MA (US)

(21) Appl. No.: 18/932,268

(22) Filed: Oct. 30, 2024

Related U.S. Application Data
(60) Provisional application No. 63/546,854, filed on Nov. 1, 2023.

(51) Int. Cl. A61B 10/02

(52) U.S. Cl. CPC A61B 10/025; A61B 2010/0208

(57) ABSTRACT

The present disclosure provides a bone harvester for harvesting a bone core from a bone. Also provided are tools operable with the bone harvester, including a pushing tool and a bone harvesting guide. The present disclosure also provides a method of processing a bone by using the bone harvester and/or other tools of the present disclosure. Bone cores having distorted cylindrical bodies are also described.

### CROSS REFERENCE TO RELATED APPLICATIONS

This application claims priority to copending U.S. provisional patent application 63/546,854 filed Nov. 1, 2023 to Liao et al., entitled “Bone Harvester and Method for Processing a Bone,” incorporated herein by reference.

### TECHNICAL FIELD

The present disclosure is related to tools for harvesting a bone core and method of processing a bone using the tool. A guide is taught to facilitate the harvesting of bone cores around a pattern at a cartilage.

### BACKGROUND OF THE INVENTION

Repairing joint cartilage defect has been challenging. In contrast to most tissues of a human body, it is difficult for a damaged cartilage tissue to heal on its own because there are no blood vessels or nerves within cartilage. A damaged cartilage therefore might develop into chronic and progressive deterioration in the joint. Once the deterioration reaches the bone layer, a patient will be suffering from severe pain and might not be able to move easily and freely. Eventually, artificial joint replacement would be the only clinical option for the patient. Therefore, an efficient approach for facilitating joint cartilage defect stabilization and healing at an early stage remains of great need to the patients and medical communities.

### SUMMARY OF THE INVENTION

In light of the foregoing, the present disclosure provides a bone harvester for harvesting a bone core, comprising a shaft having a lumen, a distal end with a blade having a distal cutting edge, a proximal end, and an engagement element that locks the relative rotation of the bone harvest and the bone core.

The present disclosure also provides a bone harvesting guide comprising a first guiding pin; a second guiding pin; and a tube configured to accommodate a bone harvester; wherein the first guiding pin, the second guiding pin, and the tube are assembled into a connected assembly whereby a longitudinal axis of the first guiding pin, a longitudinal axis of the second guiding pin, and a longitudinal axis of the tube are substantially parallel to each other; and wherein, in a cross-section of the bone harvesting guide, a center of the first guiding pin, a center of the second guiding pin, and a center of the tube form vertexes of an equilateral triangle.

The present disclosure also provides a kit for harvesting a bone core, comprising: a bone harvester of the present disclosure; and a pushing tool, wherein the pushing tool comprises a pushing rod having a shape matching the lumen defined by the shaft.

The present disclosure also provides a kit for harvesting a bone core, comprising: a bone harvesting guide and a bone harvester. The bone harvesting guide comprises a first guiding pin, a second guiding pin, and a tube, wherein the first guiding pin, the second guiding pin, and the tube are assembled whereby a longitudinal axis of the first guiding pin, a longitudinal axis of the second guiding pin, and a longitudinal axis of the tube are substantially parallel to each other; and wherein, in a cross-section of the bone harvesting guide, a center of the first guiding pin, a center of the second guiding pin, and a center of the tube form vertexes of an equilateral triangle. The bone harvester comprises a shaft having a lumen, a distal end with a blade having a distal cutting edge, and a proximal end. The tube of the bone harvesting guide is configured to accommodate the bone harvester.

The present disclosure also provides a method for processing a bone. The method comprises inserting a bone harvester of the present disclosure into a bone whereby at least a portion of the engagement element is within the bone and the bone core is within the lumen; rotating the bone harvester to rotate the bone core relative to the bone to shear the base of the bone core from the bone; and withdrawing the bone harvester thereby obtaining a harvested bone core.

The present disclosure also provides a bone core comprising a distorted cylindrical body wherein the distorted cylindrical body has a non-circular cross section perpendicular to the cylindrical axis corresponding to an accretion to or removal from the corresponding circular form.

### BRIEF DESCRIPTION OF THE DRAWINGS

FIG. 1A illustrates a bone harvester according to an embodiment of the present disclosure.

FIG. 1B is a photo of a bone harvester according to an embodiment of the present disclosure.

FIG. 1C illustrates a sectional view of a bone harvester according to another embodiment of the present disclosure.

FIG. 2A is a photo showing a distal end of a bone harvester according to an embodiment of the present disclosure.

FIG. 2B is a photo showing an enlarged view of the blade, the cutting edge, and the open slot of the bone harvester shown in FIG. 2A.

FIG. 3A illustrates a sectional view of a distal end of a bone harvester having an open slot as an engagement element.

FIG. 3B illustrates a side view of the distal end of the bone harvester shown in FIG. 3A.

FIG. 3C illustrates a front view of the bone harvester shown in FIG. 3A observed from the distal end of the bone harvester.

FIG. 3D illustrates a sectional view of a distal end of a bone harvester having a notch as an engagement element.

FIG. 3E illustrates a front view of the bone harvester shown in FIG. 3D observed from the distal end of the bone harvester.

FIG. 3F illustrates a sectional view of a distal end of a bone harvester having a plurality of internal slots as an engagement element.

FIG. 3G illustrates a front view of the bone harvester shown in FIG. 3F observed from the distal end of the bone harvester.

FIG. 3H illustrates a sectional view of a distal end of a bone harvester having a collar as an engagement element.

FIG. 3I illustrates a front view of the bone harvester shown in FIG. 3H observed from the distal end of the bone harvester.

FIG. 3J illustrates a front view of a distal end of a bone harvester having a plurality of flat faces as an engagement element.

FIG. 3K illustrates a front view of a bone harvester having multiple engagement elements.

FIG. 4A illustrates a side view showing how the cutting edge and the open slot according to an embodiment of the present invention interface a bone.

FIG. 4B illustrates a sectional view of FIG. 4A.

FIG. 5A illustrates a rear view of a handle module according to an embodiment of the present disclosure observed from a proximal end thereof.

FIG. 5B illustrates a perspective view of the handle module of FIG. 5A showing portions of the two rods mounted with the body of the handle module and two screw holes inside the body.

FIG. 6A illustrates a bone harvesting guide according to an embodiment of the present disclosure.

FIG. 6B illustrates a cross-sectional view of the bone harvesting guide of FIG. 6A.

FIG. 6C is a photo showing a prototype of a bone harvesting guide according to an embodiment of the present disclosure.

FIG. 7A illustrates a bone harvester and a bone harvesting guide according to the present disclosure from a side view.

FIG. 7B illustrates an assembly of the bone harvester and the bone harvesting guide of FIG. 7A.

FIG. 8A illustrates a cross-sectional view of a pushing tool according to an embodiment of the present disclosure.

FIG. 8B illustrates a cross-sectional view showing the pushing tool according to an embodiment of the present disclosure is assembled with a bone harvester.

FIG. 9A illustrates a pushing tool according to an embodiment of the present disclosure.

FIG. 9B illustrate a leading rod configured to use together with a pushing tool according to an embodiment of the present disclosure.

FIG. 9C illustrates assembly of the pushing tool of FIG. 9A and the leading rod of FIG. 9B with a bone harvester of an embodiment of the present disclosure.

FIG. 10 illustrates steps of a method according to an embodiment of the present disclosure.

FIG. 11A illustrates a first hole is left on the bone after a first bone core is harvested according to an embodiment of a method of the present disclosure.

FIG. 11B illustrates inserting a first guiding pin of a bone harvesting guide of the present disclosure into the first hole shown in FIG. 11A.

FIG. 11C illustrates a cross-sectional view showing that the first guiding pin 610 is inside the first hole.

FIG. 11D illustrates that a bone harvester is assembled with the tube of the bone harvesting guide while the first guiding pin of the harvesting guide is inside the first hole.

FIG. 11E illustrates a cross-sectional view showing that the bone harvester is inside the bone with its blade cut into the bone, and the first guiding pin is maintained inside the first hole.

FIG. 11F illustrates that two holes are left on the bone after the bone harvester is withdrawn and the second bone core is collected.

FIG. 11G illustrates a cross-sectional view showing that two holes are left on the bone.

FIG. 11H illustrates that the first guiding pin and the second guiding pin of the bone harvesting guide are inserted into the first hole and the second hole on the bone respectively.

FIG. 11I illustrates that a bone harvester is assembled with the tube of the bone harvesting guide while the first guiding pin and the second guiding pin of the harvesting guide are inside the first hole and second hole respectively.

FIG. 11J illustrates that three holes are left on the bone after the bone harvester is withdrawn and the third bone core is collected.

FIG. 11K shows a photo of a bone from which three bone cores were collected using a bone harvester and a bone harvesting guide according to an embodiment of the present disclosure. Three equidistant holes were left on the bone after the bone cores were collected.

FIG. 12 shows photos of a working example performing the method of processing a bone according to an embodiment of the present disclosure.

FIG. 13A is a photo showing a side view of a bone core collected using a bone harvester according to the present disclosure.

FIG. 13B is a photo showing a top view of the bone core shown in FIG. 13A.

### DETAILED DESCRIPTION

To make the structure and characteristics as well as the effectiveness of the present disclosure further understood and recognized, the detailed description of the present disclosure is provided as follows along with embodiments and accompanying figures. It is important to note that the present disclosure may be embodied in many different forms and are not limited to the embodiments set forth herein.

Unless indicated otherwise in context, the term “comprise,” “include,” or “have” used herein is intended to describe the presence of state features, integers, steps, operations, members, components and/or a combination thereof but not preclude the possibility of the presence or addition of one or more other features, integers, steps, operations, members, components, or a combination thereof. Only the transitional phrases “consisting of” and “consisting essentially of” shall be closed or semi-closed transitional phrases, respectively.

Tools are described to provide consistent and efficient harvesting of bone plugs (i.e., bone cores). The tools can be effectively used in procedures for harvesting plugs of bone tissue, which can be particularly useful for harvesting plugs encompassing regions with cartilage that may be damaged or with healthy cartilage for transplanting to other regions. The bone harvester has a leading cutting edge and an open lumen for accepting the bone plug. A noncircular cross section allowing for engaging the bone plug and applying torque to sever the bottom connection to the underlying bone while the walls of the plug are supported by the tool. The harvested bone plugs can be effective for preparing treatment of a region experiencing cartilage damage or wear. A cutting guide can facilitate the harvesting process for performing a series of appropriately spaced holes, such as equally spaced holes, to treat an appropriate region. The procedure can be performed in a low invasive format, such as through an arthroscopy procedure.

The bone tools of the present disclosure, including the bone harvester, handle module, bone harvester guide, and/or pushing tool, are not limited to any specific treatment procedure and can be helpful for any procedure where harvesting of a bone plug is indicated. Some previous bone harvesting tools relied on rocking the plug to disconnect the plug from the underlying tissue. See, for example, U.S. Pat. No. 6,592,588 to Bobic et al., entitled “Apparatus for Osteochondral Autograft Transplantation,” incorporated herein by reference. Such a harvesting tool can damage the plug during removal. More complex structures have been proposed to cut the lower edge of the plug. See U.S. Pat. No. 11,432,825 to Song et al., entitled “Nondestructive Autograft Extracting Device for Autologous Osteochondral Transplantation,” incorporated herein by reference. The tools described herein provide for simple use and design while allowing for twisting motion to provide removal of the plug by shearing the lower surface of the plug. Examples demonstrate clean removal of plugs reproducibly without damage.

The bone harvesters can be effective to form a corresponding cavity (e.g., a corresponding hole) at a selected location. The cavity can provide access to underlying tissue as well as providing a location for placement of material to support tissue healing and regeneration.

As is conventional in the art, the term “distal” as used herein shall mean away from a body of a user while operating an invention of the present disclosure. The term “proximal,” while being used in contrast to “distal”, shall mean closer towards a body of a user operating an invention of the present disclosure and/or away from the “distal” end.

### Treatment of Cartilage Defect

Clinical strategies for treating a cartilage defect can involve facilitating a self-healing process. Suitable approaches include, for example, autologous chondrocyte implantation (ACI), Mosaicplasty procedure, and microfracture. The ACI approach facilitates the self-healing process by taking out a tissue of cartilage from a damaged site of a patient, proliferating chondrocytes in vitro under strict conditions, and then implanting the proliferated chondrocytes back to the damaged site. The bone harvester can be used to remove the damaged cartilage tissue. This approach is costly and inconvenient because of the in vitro proliferation process and because at least two surgeries are needed to complete one course of treatment.

The concept of Mosaicplasty procedure is to implant an autologous healthy bone core to a damaged site. That approach requires drilling a hole at the damaged site and implanting a bone core, which is removed from a healthy region of a joint, usually from a region that is less exposed to weight bearing. The bone harvesters described herein can be used for removing bone at both locations used in the Mosaicplasty procedure. The bone core required for this approach is typically about 8 mm in diameter and 20 mm in length.

Microfracture is the current standard of treatment. This approach drills several holes at the damaged site of the joint, each has a diameter of about 2 mm and a depth of about 4 mm. The depth of the hole should be deep enough to reach subchondral tissue so that bone marrow, which is rich in stem cells, can flow out to the damaged cartilage. The holes can be formed using the harvesters described herein. The stem cells can then form a new layer of cartilage-like tissue slowing down the degeneration of the joint and insulating the nerves of the bone tissue thereby reducing the pain.

### Bone Harvester

Several embodiments are described with respect to the bone harvester. The different embodiments invoke a common conceptual idea, in which the bone harvester forms a segment of cut tissue with a non-circular cross section. The cutter is delivered laterally downward into the tissue. Since the cut tissue has a non-circular cross section, twisting of the cutter after placement can shear the bottom of the plug from the underlying tissue to have a cleanly severed plug that is removed by pulling up on the bone harvester after the twisting motion. A tool can then be used to push the bone core from the harvester lumen. In exemplified embodiments, the shaft of the bone harvester has a space along the circumference such that the cut bone plug has a ledge to provide the torque upon twisting. Alternative embodiments have a protuberance into the lumen to result in a missing piece in the plug relative to a cylinder that similarly provides a non-circular cross section. Generally, combinations and variations in these principles can be correspondingly used.

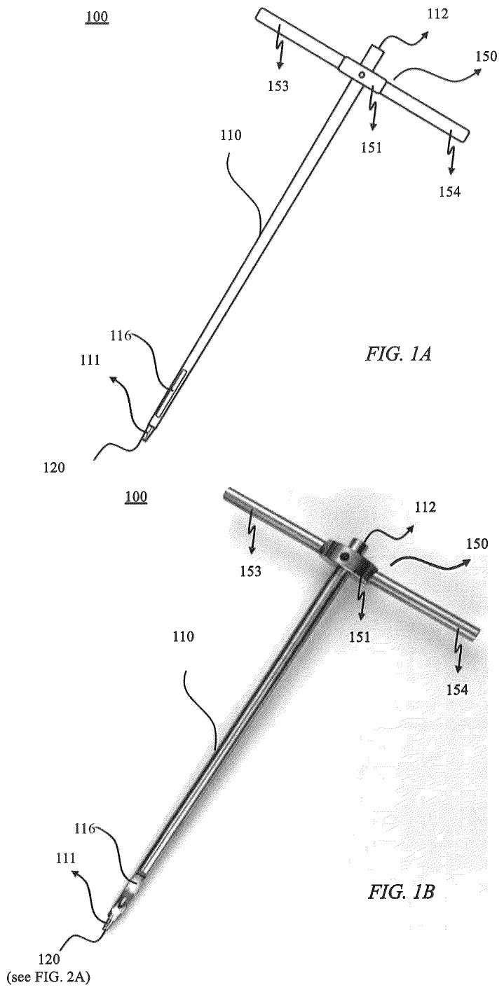

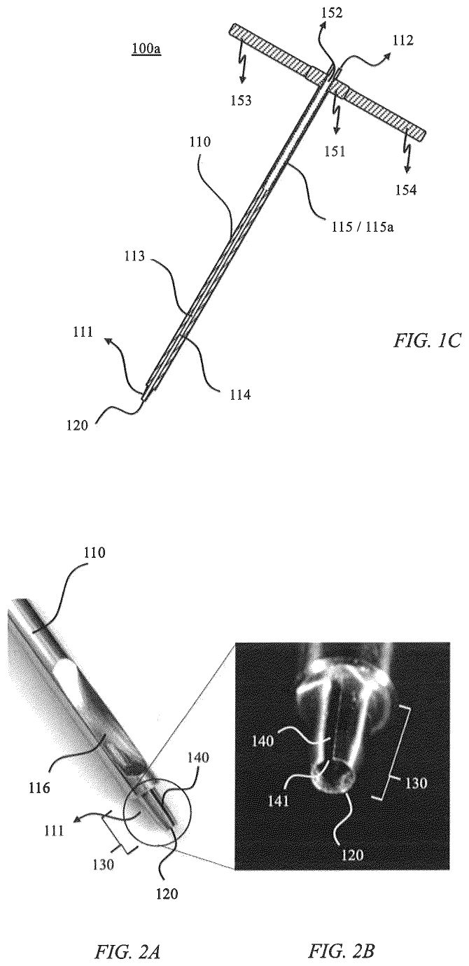

One aspect of the present disclosure provides an embodiment of a bone harvester for harvesting a bone core with an open slot in the wall of the cutting surface along the lumen. See FIG. 1A and FIG. 1B. FIG. 1A illustrates a bone harvester 100 according to an embodiment of the present disclosure, and FIG. 1B shows a photo of an actual bone harvester according to an embodiment of the present disclosure. The bone harvester 100 comprises a shaft 110 having a lumen 113, a distal end 111 with a blade 130 having a distal cutting edge 120, a proximal end 112, and an engagement element that locks the relative rotation of the bone harvester and the bone core. As used herein, “lock” of the relative rotation of the bone harvester and the bone core refers to a restriction of the rotation of the bone harvester relative to the bone core (or the bone) by the engagement element. Thus, the bone harvester can be rotated with the bone core, and the rotation applies torque to snap the root of the bone core from the bone.

The engagement element is an open slot 140 in this example. In other embodiments, the engagement element can be a notch, a flat face, an internal slot substantially parallel to a longitudinal axis of the shaft, or a collar, which are described in detail in the following paragraphs. In some embodiments, the engagement element can be a plurality of an open slot, a notch, a flat face, an internal slot substantially parallel to a longitudinal axis of the shaft, or a collar, or a combination thereof. The shaft 110 comprises a distal end 111 and a proximal end 112. The distal end 111 is identified as the end of the shaft 110 that is away from a user while operating the bone harvester 100 for its intended purpose, and the proximal end 112 is identified as the end that is proximal to the user.

Shaft of the bone harvester. In some embodiments, at least a portion of the shaft is a hollow lumen that can approximate a cylinder. While a cylinder configuration is particularly suitable for the bone harvester to rotate within a bone, a cylindrical configuration does not provide torque to shear the bone plug from the underlying tissue. Viewing the harvester from its outer surface, the hollow cylinder refers to the outer shape of the shaft. The inner cross-section of the shaft can be generally circular for rotating the cutter to form the bone plug, but the presence of the engagement element alters the circular shape to allow for coupling the rotational motion of the shaft and the bone plug. The inner cross-section of the shaft can be a distorted circular shape with a notch or notches in shapes, such as an oval or circular with a bump, and/or a flat face(s) that can be used to introduce the non-circular cross section of the plug. If the outer wall of the shaft has an open slot to form a non-circular cross section for the bone plug, the circular cross section has a gap at the location of the slot. Various alterations of the circular nature of the inner cross section follow from the discussion of other specific embodiments. In some embodiments, a portion of the shaft is a hollow cylinder, while another portion thereof is a rectangular cuboid. In such embodiments, the portion of the rectangular cuboid might not be the portion of the shaft that is inserted into a bone. In some embodiments, the entire shaft 110 is a hollow cylinder as shown in FIG. 1C. In comparison, in the embodiment shown in FIG. 1A, the lumen of the shaft 110 only extends from the distal end 111 of the shaft 110 to the window 116. Other parts of the shaft 110 is solid.

In some embodiments, the shaft has a length from about 50 mm to about 200 mm, from about 50 mm to about 150 mm, 100 mm to about 200 mm, or from about 100 mm to about 150 mm. In some embodiments, the blade has a length from about 1 mm to about 20 mm, from about 2 mm to about 20 mm, from about 5 mm to about 20 mm, from about 7 mm to about 20 mm, from about 10 mm to about 20 mm, from about 1 mm to about 15 mm, from about 2 mm to about 15 mm, from about 5 mm to about 15 mm, from about 7 mm to about 15 mm, from about 10 mm to about 15 mm, from about 1 mm to about 2 mm, from about 1 mm to about 3 mm, from about 1 mm to about 5 mm, from about 1 mm to about 10 mm, from about 2 mm to about 3 mm, from about 2 mm to about 5 mm, from about 2 mm to about 10 mm, or from about 5 mm to about 10 mm.

The term “bone core” is interchangeable with the term “bone plug,” “plug,” or “core.” In some contexts, if the size of the bone core is smaller, it may be more difficult to remove from a bone without breaking the structural integrity thereof. The bone harvester of the present disclosure is configured and designed to collect a bone core of any size without damaging the bone core during removal. In some embodiments, the bone core can be collected by using the bone harvester of the present disclosure has a length of about 2, 2.5, 3, 3.5, 4, 4.5, 5, 5.5, 6, 6.5, 7, 7.5, 8, 9, 10, 11, 12 mm, or a range therein. In some embodiments, the bone core can be collected by using the bone harvester of the present disclosure has a length of about 2 to 12 mm, 3 to 12 mm, 4 to 12 mm, 5 to 12 mm, 6 to 12 mm, 7 to 12 mm, 8 to 12 mm, 9 to 12 mm, 10 to 12 mm, 11 to 12 mm, 2 to 11 mm, 3 to 11 mm, 4 to 11 mm, 5 to 11 mm, 6 to 11 mm, 7 to 11 mm, 8 to 11 mm, 9 to 11 mm, 10 to 11 mm, 2 to 10 mm, 3 to 10 mm, 4 to 10 mm, 5 to 10 mm, 6 to 10 mm, 7 to 10 mm, 8 to 10 mm, 9 to 10 mm, 4 to 9 mm, 5 to 9 mm, 6 to 9 mm, 7 to 9 mm, 8 to 9 mm, 2 to 4 mm, 3 to 4 mm, 4 to 5 mm, 4 to 6 mm 4 to 7 mm, 4 to 8 mm, 5 to 6 mm 5 to 7 mm, or 5 to 8 mm.

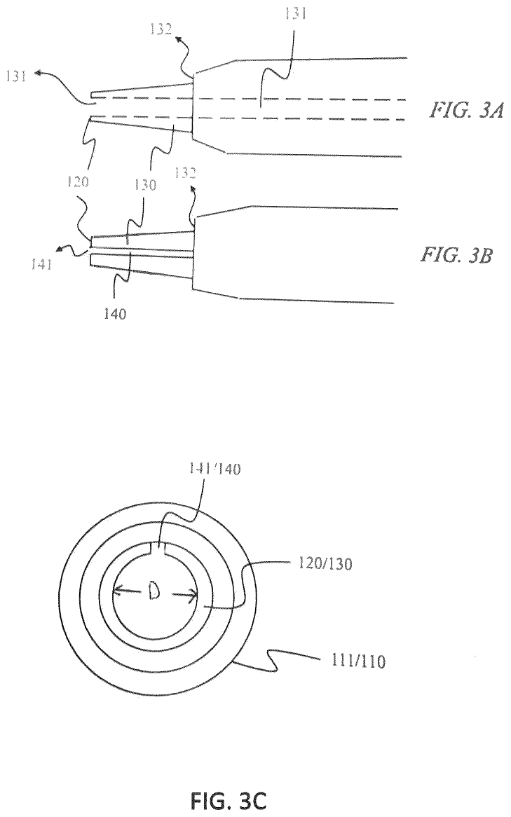

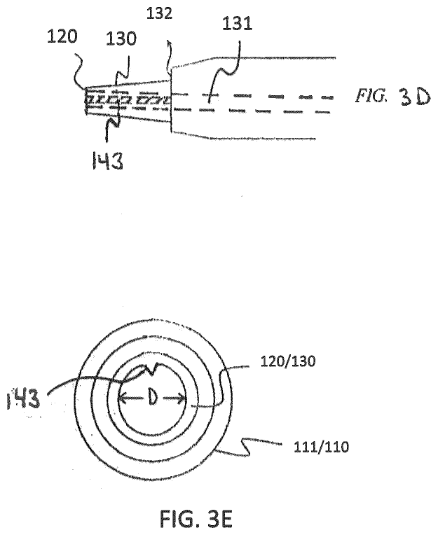

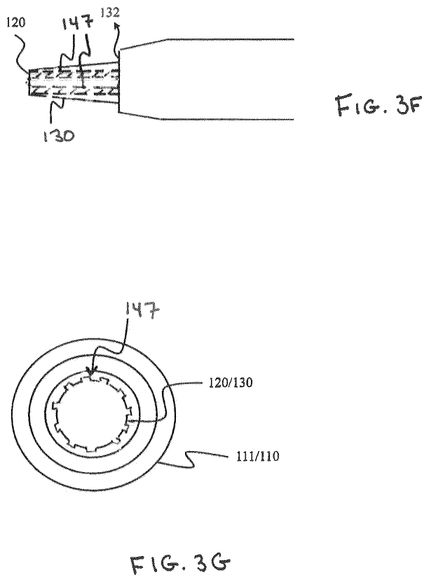

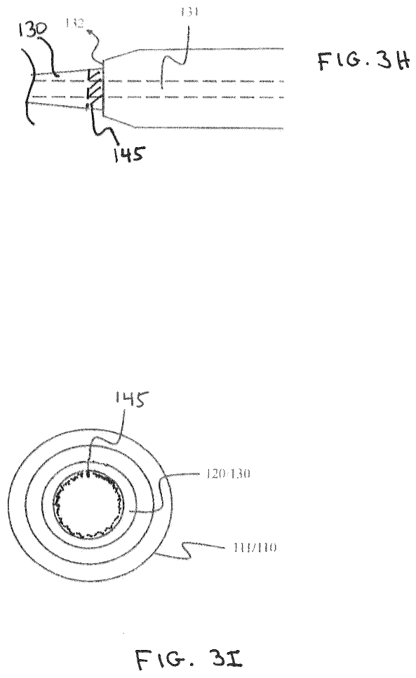

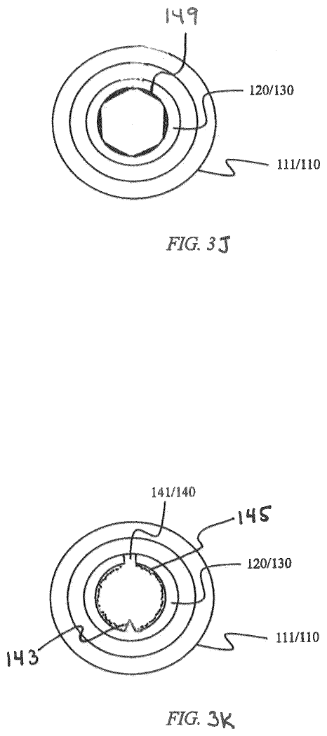

The inner lumen of the bone harvester where the bone core is collected can be characterized by a diameter which is defined by a circle where non-circular modifications are not considered. In some embodiments, the lumen extends from the distal end to the proximal end of the shaft. This inner diameter of the bone harvester roughly corresponds to the diameter of the removed plug where the diameter is referenced to a cylindrical rendition of the plug with the non-circular cross-sectional features not considered. See “D” in FIGS. 3C and 3E.

In some embodiments, the bone core can be collected by using the bone harvester of the present disclosure has a diameter of about 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12 mm, or a range therein. In some embodiments, the diameter is about 0.75 to 12 mm, 1 to 12 mm, 1.2 to 12 mm, 1.5 to 12 mm, 2 to 12 mm, 3 to 12 mm, 4 to 12 mm, 5 to 12 mm, 6 to 12 mm, 7 to 12 mm, 8 to 12 mm, 9 to 12 mm, 10 to 12 mm, 11 to 12 mm, 1 to 9 mm, 1.2 to 9 mm, 1.5 to 9 mm, 2 to 9 mm, 3 to 9 mm, 4 to 9 mm, 5 to 9 mm, 6 to 9 mm, 7 to 9 mm, 8 to 9 mm, 1 to 6 mm, 1.2 to 6 mm, 1.5 to 6 mm, 2 to 6 mm, 3 to 6 mm, 4 to 6 mm, 5 to 6 mm, 1 to 1.2 mm, 1 to 1.5 mm, 1 to 2 mm, 1 to 2.5 mm, 1 to 3 mm, 1 to 4 mm, 1 to 5 mm, 1.2 to 1.5 mm, 1.2 to 2 mm, 1.2 to 2.5 mm, 1.2 to 3 mm, 1.2 to 4 mm, 1.2 to 5 mm, 1.5 to 2 mm, 1.5 to 2.5 mm, 1.5 to 3 mm, 1.5 to 4 mm, or 1.5 to 5 mm. A person of ordinary skill in the art will recognize that additional ranges of bone core dimensions within the explicit ranges above are contemplated and are within the present disclosure.

FIG. 1C illustrates a sectional view of another embodiment of bone harvester 100a of the present disclosure. The shaft 110 defines a lumen 113, which is configured to accommodate a bone tissue during operation. In the depicted embodiment, the shaft 110 has a same cross-sectional area (e.g., has a same inner diameter) along a longitudinal axis thereof, which is convenient for manufacture.

In some embodiments, as the one shown in FIG. 1C, the shaft 110 comprises a smooth inner surface 114 at a first portion thereof. The smooth inner surface 114 is positioned proximal to the distal end 111 of the shaft so that there is less friction between the lumen 113 and a collected bone core. The shaft 110 can also have a threaded inner surface 115 at a second portion thereof. The threaded inner surface 115 is positioned proximal to the proximal end of the shaft and configured to couple with a tool for removing a collected bone core from the lumen 113, which is described below.

Referring back to FIG. 1A and FIG. 1B, shaft 110 further comprises a window 116. In some embodiments, the window 116 is positioned proximal to the blade 130 and comprises an opening on a side portion of the shaft to expose a portion of the lumen 113. The window 116 provides visibility of the bone core collected within the lumen 113. In some embodiments, the window 116 can also be served as an exit for removing a collected bone core from the lumen 113. Therefore, window 116 can be positioned appropriately proximal to the distal end 111 of the shaft 110. The size of the window 116 is not limited. In some embodiments, the window 116 should be sufficiently wide for better visibility or for the convenience to remove a collected bone core from the lumen 113. On the other hand, the window 116 should not be too wide to weaken the overall structural integrity of the bone harvester 100.

Cutting edge and engagement element. A commonly encountered problem while removing a bone core from a bone is that, while a cutting edge of a bone harvester successfully cuts into a bone and some bone tissue is retained within a lumen of a bone harvester, the root of the retained bone tissue is still firmly connected with the bone. Therefore, simply withdrawing the bone harvester does not necessarily retrieve a bone core. A medical practitioner often needs to move the bone harvester within the bone back and forth between vertical and an oblique angle to try to break the root of the retained bone tissue from the bone. Moving the bone harvester within the bone is usually inevitable but increases the risk of unnecessary harm to the bone. The bone harvester of the present disclosure is designed to have an engagement element, such as an open slot, that provides advantages of removing a collected bone core from a bone.

The shaft of the bone harvester comprises a cutting edge positioned on the distal end of the shaft. The cutting edge is configured to facilitate the insertion of the bone harvester into a bone, such as by pushing or pounding with a mallet or the like. The shaft of the present disclosure also comprises an engagement element on a side or a portion of a side thereof. In some embodiments the engagement element comprises an open slot. An “open slot” used herein is a slot that has a side opening that extends from the distal cutting edge toward the proximal end of the shaft. As shown in FIG. 2A, a cutting edge 120 is positioned on the distal end 111 of the shaft 110, and an open slot 140 is positioned on a side of the shaft 110 and has its side opening 141 at the cutting edge 120 (see also FIG. 2B). The open slot 140 comprises a longitudinal axis that is substantially parallel to a longitudinal axis of the shaft 110. The open slot 140 can be viewed as extending from the cutting edge 120 into the shaft toward the proximal end 112 thereof (FIGS. 2A-2B and FIG. 3B).

In some embodiments, as shown in FIGS. 2A-2B and FIGS. 3A-3K, the shaft 110 comprises a blade 130 at its distal end 111, and the cutting edge 120 is positioned at a tip of the blade 130. In some embodiments, blade 130 can be a circular blade comprising a longitudinal through-bore 131 connecting to the lumen 113 of shaft 110 (FIGS. 3A-3I and 3K). In other embodiments, blade 130 comprises one or more faces 149. (FIG. 3J.) The blade 130 will be described in more details in the following paragraphs. Referring to FIGS. 3A-3C, open slot 140 is positioned on a side of blade 130 with its side opening 141 at the cutting edge 120. The open slot 140 can be viewed as extending from the cutting edge 120 into the shaft 110, specifically the blade 130.

In some embodiments with an open slot, a side of open slot 140 is not a cutting edge, and, none of the sides of the open slot 140 may be a cutting edge. Open slot 140, with its side opening 141 positioned at cutting edge 120, creates a non-cutting portion of the contact surface. See FIG. 3C for a front view of the bone harvester from distal end 111 of shaft 110. The inner circle shows cutting edge 120 on the tip of blade 130. In this front view, side opening 141 of open slot 140, being positioned at the cutting edge 120, is shown as the discontinued portion of the circle with a gap corresponding to open slot 140.

The length of the open slot of the present disclosure is not limited. In some embodiments, without wishing to be bound by theories, in order to better facilitating retrieving a bone core, the length of the open slot can be about at least 10%, 20%, 30%, 40%, 50%, 60%, 70%, 80%, 90%, 95%, 100%, or a range therein of a length of the bone core. In some embodiments, the length of the open slot is about 10 to 100%, 20 to 100%, 30 to 100%, 40 to 100%, 50 to 100%, 60 to 100%, 70 to 100%, 80 to 100%, 90 to 100%, 95 to 100%; or 10 to 95%, 20 to 95%, 30 to 95%, 40 to 95%, 50 to 95%, 60 to 95%, 70 to 95%, 80 to 95%, 90 to 95%, 30 to 80%, 40 to 80%, 50 to 80%, 60 to 80%, 70 to 80%, 40 to 50%, 40 to 60%, 40 to 70%, 50 to 60%, 50 to 70%, 50 to 80%, 50 to 90%, 50 to 95%, 60 to 70%, 60 to 80% 60 to 90%, or 60 to 95%, of a length of the bone core. In some embodiments, the length of the open slot is about 0.1, 0.2, 0.5, 1, 2, 2.5, 3, 3.5, 4, 4.5, 5, 5.5, 6, 6.5, 7, 7.5, 8, 9, 10, 11, 12 mm, or a range therein. In some embodiments, the open slot has a length of about 0.1 to 12 mm, 0.5 to 12 mm, 1 to 12 mm, 2 to 12 mm, 3 to 12 mm, 4 to 12 mm, 5 to 12 mm, 6 to 12 mm, 7 to 12 mm, 8 to 12 mm, 9 to 12 mm, 10 to 12 mm, 11 to 12 mm, 2 to 11 mm, 3 to 11 mm, 4 to 11 mm, 5 to 11 mm, 6 to 11 mm, 7 to 11 mm, 8 to 11 mm, 9 to 11 mm, 10 to 11 mm, 2 to 10 mm, 3 to 10 mm, 4 to 10 mm, 5 to 10 mm, 6 to 10 mm, 7 to 10 mm, 8 to 10 mm, 9 to 10 mm, 4 to 9 mm, 5 to 9 mm, 6 to 9 mm, 7 to 9 mm, 8 to 9 mm, 2 to 4 mm, 3 to 4 mm, 4 to 5 mm, 4 to 6 mm 4 to 7 mm, 4 to 8 mm, 5 to 6 mm 5 to 7 mm, or 5 to 8 mm.

The width of the open slot is not limited. In some embodiments, without wishing to be bound by theories, in order to better facilitating retrieving a bone core, the width of the open slot can be about at least 1, 2, 3, 4, 5, 6, 7, 8, 9, 10, 15, 20, 30%, or a range therein of a circumference of a bone core to be collected. In some embodiments, the width of the open slot is 1 to 2%, 1 to 3%, 1 to 5%, 1 to 6%, 1 to 7%, 1 to 8%, 1 to 9%, 1 to 10%, 1 to 15%, 1 to 20%, 1 to 30%, 5 to 10%, 5 to 15%, 5 to 20%, 5 to 30%, 10 to 15%, 10 to 20%, or 10 to 30% of a circumference of a bone core to be collected. In certain embodiments, the width of the open slot can be about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.5, 3, 3.5, 4, 4.5, 5, 6, 7, 8, 9, 10, or 12 mm, or a range therein. In certain embodiments, the width of the open slot is 0.1 to 0.5 mm, 0.1 to 1 mm, 0.1 to 1.5 mm, 0.1 to 2 mm, 0.1 to 2.5 mm, 0.1 to 3 mm, 0.1 to 3.5 mm, 0.1 to 4 mm, 0.1 to 6 mm, 0.1 to 7 mm, 0.1 to 8 mm, 0.1 to 9 mm, 0.1 to 10 mm, 0.1 to 12 mm, 0.2 to 0.5 mm, 0.2 to 1 mm, 0.2 to 1.5 mm, 0.2 to 2 mm, 0.2 to 2.5 mm, 0.2 to 3 mm, 0.2 to 3.5 mm, 0.2 to 4 mm, 0.2 to 6 mm, 0.2 to 7 mm, 0.2 to 8 mm, 0.2 to 9 mm, 0.2 to 10 mm, 0.2 to 12 mm, 0.3 to 0.6 mm, 0.3 to 1 mm, 0.3 to 1.5 mm, 0.3 to 2 mm, 0.3 to 2.5 mm, 0.3 to 3 mm, 0.3 to 3.5 mm, 0.3 to 4 mm, 0.3 to 6 mm, 0.3 to 7 mm, 0.3 to 8 mm, 0.3 to 9 mm, 0.3 to 10 mm, 0.3 to 12 mm, 1 to 1.5 mm, 1 to 2 mm, 1 to 2.5 mm, 1 to 3 mm, 1 to 3.5 mm, 1 to 4 mm, 1 to 6 mm, 1 to 7 mm, 1 to 8 mm, 1 to 9 mm, 1 to 10 mm, 1 to 12 mm, 4 to 6 mm, 4 to 7 mm, 4 to 8 mm, 4 to 9 mm, 4 to 10 mm, or 4 to 12 mm. A person of ordinary skill in the art will recognize that additional ranges of open slot dimensions and parameters within the explicit ranges above are contemplated and are within the present disclosure.

In some embodiments, as shown in FIGS. 3D-3E, the engagement element comprises a notch 143 positioned on a side of blade 130. In some embodiments, as shown in FIGS. 3D-3E, notch 143 is continuous with cutting edge 120 and extends from cutting edge 120 to step 132. In some embodiments, a plurality of notch 143 may be provided. Notch 143 may have a triangular cross-section as depicted in FIG. 3E. In some embodiments, features of notch 143 such as the cross-sectional dimensions (base, height), the cross-sectional shape (triangular, trapezoidal, rectangular, etc), the length, the number of notch 143 elements and/or the spacing of multiple notch 143 elements can be chosen to achieve desired bone harvesting outcomes. In some embodiments, multiple notch 143 elements can be provided with each notch 143 having independently chosen features. Faces of notch 143 engage the bone plug as the bone harvester is rotated to provide shear at the bottom surface of the bone plug to release the bone plug from the associated tissue.

In some embodiments, the notch has a dimension of at least about 1, 1.5, 2, 2.5, 3, 3.5, 4, 4.5, 5, 5.5, or 6 mm, or a range therein. In some embodiments, the notch has a dimension of from about 1 to about 6 mm, from about 2 to about 6 mm, from about 3 to about 6 mm, from about 4 to about 6 mm, from about 5 to about 6 mm, from about 2 to about 3 mm, from about 2 to about 4 mm, from about 2 to about 5 mm, from about 3 to about 4 mm, from about 3 to about 5 mm, or from about 3 to about 6 mm.

In some embodiments, as shown in FIGS. 3F-3G, the engagement element comprises a plurality of internal slots 147 positioned on a side of blade 130. Internal slots 147 form openings at cutting edge 120 and extend into blade 130 without extending through the full thickness of the wall of the bone harvester. In contrast to open slot 140 with side opening 141, internal slots 147 do not form side openings. With this design, the walls of the bone plug are fully severed from the adjacent tissue while providing engagement surfaces to rotate the bone plug with the bone harvester. In some embodiments, as shown in FIGS. 3F-3G, internal slots 147 extend from cutting edge 120 to step 132. Internal slots 147 may have any convenient cross-sectional shape, such as a rectangular cross-section as depicted in FIG. 3G. In some embodiments, the cross-sectional dimensions (base, height) of internal slots 147, the cross-sectional shape (triangular, trapezoidal, rectangular, etc.) of internal slots 147, the length of internal slots 147, the number of internal slots 147 and/or the spacing of internal slots 147 can be chosen to achieve desired bone harvesting outcomes. In some embodiments, individual internal slots 147 can be provided with independently chosen features.

In some embodiments, the length of the internal slot is about 0.1, 0.2, 0.5, 1, 2, 2.5, 3, 3.5, 4, 4.5, 5, 5.5, 6, 6.5, 7, 7.5, 8, 9, 10, 11, 12 mm, or a range therein. In some embodiments, the internal slot has a length of about 0.1 to 12 mm, 0.5 to 12 mm, 1 to 12 mm, 2 to 12 mm, 3 to 12 mm, 4 to 12 mm, 5 to 12 mm, 6 to 12 mm, 7 to 12 mm, 8 to 12 mm, 9 to 12 mm, 10 to 12 mm, 11 to 12 mm, 2 to 11 mm, 3 to 11 mm, 4 to 11 mm, 5 to 11 mm, 6 to 11 mm, 7 to 11 mm, 8 to 11 mm, 9 to 11 mm, 10 to 11 mm, 2 to 10 mm, 3 to 10 mm, 4 to 10 mm, 5 to 10 mm, 6 to 10 mm, 7 to 10 mm, 8 to 10 mm, 9 to 10 mm, 4 to 9 mm, 5 to 9 mm, 6 to 9 mm, 7 to 9 mm, 8 to 9 mm, 2 to 4 mm, 3 to 4 mm, 4 to 5 mm, 4 to 6 mm 4 to 7 mm, 4 to 8 mm, 5 to 6 mm 5 to 7 mm, or 5 to 8 mm.

In certain embodiments, the width of the internal slot can be about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.5, 3, 3.5, 4, 4.5, 5, 6, 7, 8, 9, 10, or 12 mm, or a range therein. In certain embodiments, the width of the internal slot is 0.1 to 0.5 mm, 0.1 to 1 mm, 0.1 to 1.5 mm, 0.1 to 2 mm, 0.1 to 2.5 mm, 0.1 to 3 mm, 0.1 to 3.5 mm, 0.1 to 4 mm, 0.1 to 6 mm, 0.1 to 7 mm, 0.1 to 8 mm, 0.1 to 9 mm, 0.1 to 10 mm, 0.1 to 12 mm, 0.2 to 0.5 mm, 0.2 to 1 mm, 0.2 to 1.5 mm, 0.2 to 2 mm, 0.2 to 2.5 mm, 0.2 to 3 mm, 0.2 to 3.5 mm, 0.2 to 4 mm, 0.2 to 6 mm, 0.2 to 7 mm, 0.2 to 8 mm, 0.2 to 9 mm, 0.2 to 10 mm, 0.2 to 12 mm, 0.3 to 0.6 mm, 0.3 to 1 mm, 0.3 to 1.5 mm, 0.3 to 2 mm, 0.3 to 2.5 mm, 0.3 to 3 mm, 0.3 to 3.5 mm, 0.3 to 4 mm, 0.3 to 6 mm, 0.3 to 7 mm, 0.3 to 8 mm, 0.3 to 9 mm, 0.3 to 10 mm, 0.3 to 12 mm, 1 to 1.5 mm, 1 to 2 mm, 1 to 2.5 mm, 1 to 3 mm, 1 to 3.5 mm, 1 to 4 mm, 1 to 6 mm, 1 to 7 mm, 1 to 8 mm, 1 to 9 mm, 1 to 10 mm, 1 to 12 mm, 4 to 6 mm, 4 to 7 mm, 4 to 8 mm, 4 to 9 mm, 4 to 10 mm, or 4 to 12 mm.

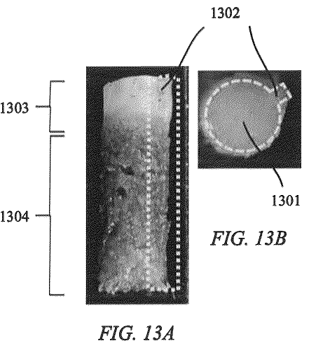

In some embodiments, as shown in FIGS. 3H-3I, the engagement element comprises collar 145 positioned along an inner circumference of blade 130 approximately adjacent to step 132. In some embodiments, collar 145 comprises a rough surface or an engraved surface. A rough surface of collar 145 may be provided by sanding or etching or similar processes. An engraved surface of collar 145 may have a pattern of grooves, such as a cross-hatch pattern or spaced-apart longitudinal lines. Collar 145 may be provided with relatively deep grooves and/or relatively high roughness to provide enhanced engagement with the bone to be harvested. In other embodiments, collar 145 may be provided with relatively shallow grooves and/or relatively low roughness to limit frictional resistance to the introduction of blade 130 while still providing supplemental friction to facilitate the shearing of the root of the bone core from the adjacent bone. Collar 145 can have a width of less than about 1 mm, about 1 to 5 mm, 2 to 3 mm, 1 to 2 mm. In some embodiments the width of collar 145 is approximately the thickness of layer of cartilage tissue 1302 (FIG. 13B).

FIGS. 3J and 3K depict further embodiments of engagement elements. FIG. 3J depicts a front view of a distal end of a bone harvester having a plurality of flat faces 149 positioned along an inner circumference of blade 130. The number of flat faces may generally be from 1 to 10, 1 to 8, 1 to 6. In some embodiments, flat faces 149 extend from cutting edge 120 to step 132 and may be optionally provided with a pattern of grooves to further enhance engagement, as described above. FIG. 3K depicts a front view of a distal end of a bone harvester having a plurality of engagement elements. In particular, FIG. 3K illustrates an embodiment having open slot 140 across from notch 143 and collar 145. A person of ordinary skill in the art will recognize that additional combinations of engagement elements, relative locations, and features thereof as described above are contemplated and are within the present disclosure.

In some embodiments, the flat face has a dimension of at least about 1, 1.5, 2, 2.5, 3, 3.5, 4, 4.5, 5, 5.5, or 6 mm, or a range therein. In some embodiments, the flat face has a dimension of from about 1 to about 6 mm, from about 2 to about 6 mm, from about 3 to about 6 mm, from about 4 to about 6 mm, from about 5 to about 6 mm, from about 2 to about 3 mm, from about 2 to about 4 mm, from about 2 to about 5 mm, from about 3 to about 4 mm, from about 3 to about 5 mm, or from about 3 to about 6 mm.

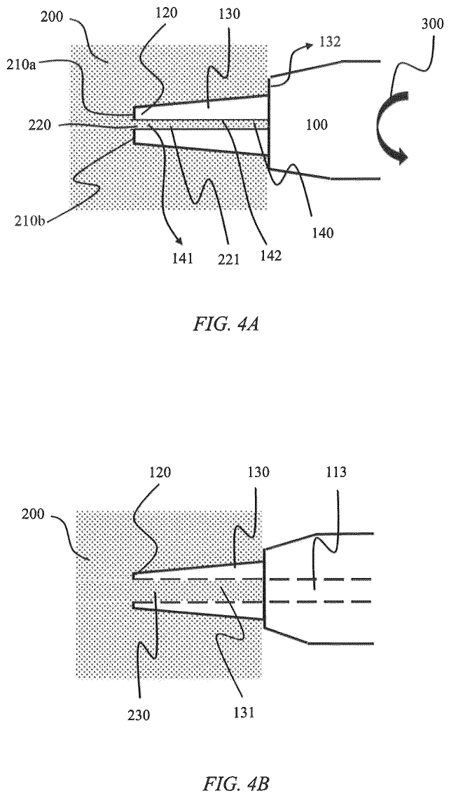

FIG. 4A illustrates the interfacing between the cutting edge 120, the open slot 140, and a bone 200. When the bone harvester 100 is inserted into a bone 200, bone tissues 210a and 210b are cut while contacting the cutting edge 120. Bone tissue 220, however, is not cut because it contacts the side opening 141 of the open slot 140 instead of a cutting edge. While some bone tissues (i.e. a bone core 230) are retained within the longitudinal through-bore 131 of the blade 130 (FIG. 4B), some of those retained bone tissues 221 are within the open slot 140. The bone tissue 221 within the open slot 140 is still connected to the bone because the bone tissue 220 is not cut. Besides the root of the bone core 230 is also still connected with the bone 220 and needed to be broken for collecting the bone core.

Next, the bone harvester 100 can be rotated (for instance, to a direction as indicated by arrow 300). While rotating the bone harvest 100, the cutting edge 120 cut the bone at the bone tissues 210a and 201b, and the bone tissue 221 within the open slot 140 is pushed by a side 142 of the open slot 140. The push snaps the bone at the site of the bone tissue 220 thereby facilitating breaking the root of the bone core 230 from the bone 220. As a result, the bone core 230 can be easily retrieved.

In some embodiments, the shaft comprises more than one open slot positioned on a side thereof (e.g., on a side of the blade thereof). In certain embodiments, the shaft comprises two open slots positioned on opposite sides thereof. In certain embodiments, the shaft comprises three open slots respectively on positioned a side of the shaft, and each open slot may be substantially equidistant from each other, although other configurations can be effective. In embodiments that the shaft comprises more than one open slot, each open slot may or may not have the same length and/or same width with one another.

With respect to the blade, as described above, blade 130 can have a circumferential shape comprising a longitudinal through-bore 131 connecting to lumen 113 of shaft 110. The longitudinal through-bore 131 is configured to retain bone tissue when the bone harvester is inserted into a bone. Depending on how deep the bone harvester is inserted into the bone, some bone tissues might enter and be retained within lumen 113. The bone tissue retained inside the longitudinal through-bore 131 is then a bone core harvested. In other words, the shape of the bone core harvested is mainly determined by the shape of the longitudinal through-bore 131. The cross-section of the longitudinal through-bore can also be other non-circular shapes. In some embodiments, the longitudinal through-bore comprises a first inner diameter, and the lumen of the shaft comprises a second inner diameter, and the first inner diameter is substantially the same as the second inner diameter. In other embodiments, the second inner diameter can be greater than the first inner diameter.

In some embodiments, an exterior of the blade 130 is tapered with a smaller outer diameter at a distal end thereof where the cutting edge 120 is positioned (FIG. 3B). In some embodiments, the outer diameter of the blade at cutting edge 120 is no more than about 1.5 mm, 1.75 mm, 2 mm, 3 mm, 4 mm, 5 mm, 6 mm, 10 mm. In some embodiments, the outer diameter of the blade at a distal end is in a range from about 1.5 mm to about 22 mm, about 2 mm to about 22 mm, about 3 mm to about 22 mm, about 2 mm to about 10 mm, about 2 mm to about 5 mm. In some embodiments, the outer diameter of the blade is from about 1.5 mm to about 22 mm, about 2 mm to about 22 mm, about 2 mm to about 10 mm, about 3 mm to about 10 mm, about 3 mm to about 5 mm, about 3 mm to about 4 mm. In some embodiments, the blade has a tapered outer diameter. In some embodiments, the blade has an outer diameter that tapers from about 1.5 mm to about 5 mm, about 1.5 mm to about 4 mm, about 2 mm to about 5 mm, about 2 mm to about 4 mm, about 3 mm to about 5 mm, about 3 mm to about 4 mm, about 4 mm to about 5 mm, about 4 mm to about 6 mm. A person of ordinary skill in the art will recognize that additional ranges of the outer diameter of the blade and diameter taper within the explicit ranges above are contemplated and are within the present disclosure. The tapered shape can facilitate the insertion of the blade 130 into a bone. In general, the wall thickness of blade 130 is relatively small to facilitate the insertion of blade 130 into a bone. (FIGS. 4A-4B). In some embodiments, a wall thickness of blade 130 is tapered with a smaller wall thickness at cutting edge 120. In some embodiments, a wall thickness of blade 130 is smoothly tapered from cutting edge 120 to step 132, with the wall thickness of the blade being smallest at cutting edge 120 and largest at the portion of the blade adjacent to step 132. One embodiment thereof is shown in FIG. 3A. In other embodiments, the blade is partially tapered in a taper region adjacent to the cutting edge, having a smaller wall thickness at the cutting edge that tapers to a larger wall thickness at a selected taper distance from the cutting edge. In such an embodiment, the portion of the blade proximal to the taper region can have a wall thickness that is the same as the larger wall thickness, which can facilitate easier insertion of the blade into the bone. A selected taper distance can be described as a distance from the cutting edge or as a percentage of the length of the blade. In some embodiments, a tapered or partially tapered shape of the exterior of the blade 130 is provided by a tapered or partially tapered wall thickness of blade 130 with the longitudinal through-bore associated with the blade having a constant inner diameter. A tapered or partially tapered wall thickness can facilitate the combination of a sharp cutting edge (i.e. via a thinner wall thickness at the cutting edge) and the distribution of forces through the blade during use (i.e. via thicker wall thicknesses away from the cutting edge). In some embodiments, thicker wall thicknesses away from the cutting edge allow for more contact area between an engagement element and a bone core. In some embodiments, a wall thickness of blade 130 can be in a range from about 0.05 mm to about 2 mm, about 0.05 mm to about 1 mm, about 0.05 mm to about 0.8 mm, about 0.1 mm to about 2 mm, about 0.1 mm to about 1 mm, about 0.1 mm to about 0.8 mm, about 0.4 to about 2 mm, about 0.4 to about 1 mm, about 0.4 mm to about 0.8 mm, about 0.5 mm to about 2 mm, about 0.5 to about 1 mm, about 0.5 to about 0.8 mm. In some embodiments, the blade has a wall thickness that tapers from about 0.05 to about 2 mm, about 0.1 to about 1 mm, about 0.2 to about 1 mm, about 0.4 to about 0.8 mm, about 0.5 to about 0.7 mm. In some embodiments, a selected taper distance can be a distance from about 0.5 mm to about 10 mm, from about 1 mm to about 10 mm, from about 1 mm to about 5 mm. In some embodiments, a selected taper distance can be no more than about 90%, 80%, 70%, 60%, 50%, 40%, 30%, or 20% of the length of the blade. A person of ordinary skill in the art will recognize that additional ranges of wall thickness and selected taper distances within the explicit ranges above are contemplated and are within the present disclosure. A person of ordinary skill in the art will also recognize that blade 130 may be sharpened at cutting edge 120. In as such, the wall thickness of blade 130 may be less than about 0.1 mm, less than about 0.05 mm, less than about 0.03 mm. In some embodiments, the blade 130 can be an extended part of the shaft 110, while in other embodiments, the blade 130 can be a structure manufactured separately and mounted (e.g., welded) on the distal end 111 of the shaft 110. In either embodiment, the shaft 110 comprises a step 132 adjacent to the blade 130, wherein the step 132 is formed by a change in outer diameter of the shaft 110. See FIGS. 3A-3B and FIG. 4A, a diameter of the shaft 110 proximal to the step 132 has a diameter larger than a diameter of the shaft 110 distal to the step 132 (e.g., at least 1 mm larger). The step 132 can serve as a marker indicating how deep the bone harvester is inserted into a bone. For example, in each time of collection, a user can insert the bone harvester until the step 132 against the surface of the bone so that the bone cores collected each time will be of similar length. In some embodiments, the step 132 has a position to form a bone core having a length from about 2 mm to about 12 mm, or any other lengths described herein.

In general, a wall thickness of shaft 110 can have a thickness that provides sufficient strength to the shaft. In some embodiments, a thicker wall thickness of shaft 110 provides for a larger outer diameter of shaft 110, which can be convenient for holding. In some embodiments, a wall thickness of shaft 110 proximal to step 132 is larger than a wall thickness of the shaft distal to step 132. In some embodiments, step 132 is coincident with an increase in the wall thickness of about 40%, 50%, 75%, 100%, 150%, 200%. In other embodiments, a wall thickness of the shaft proximal to step 132 is larger than the largest value of a wall thickness of blade 130. In other embodiments, such as those in which the longitudinal through-bore associated with the shaft is larger than the longitudinal through-bore associated with the blade, a wall thickness of shaft 110 proximal to step 132 is about the same as a wall thickness of the blade adjacent to step 132. In other embodiments, the wall thickness of shaft 110 is tapered with a larger thickness at the proximal end to provide an outer diameter that correspondingly tapers. (FIGS. 1A and 1B.) In other embodiments, the wall thickness of the shaft tapers from a first wall thickness at step 132 to a final wall thickness a selected distance from step 132. In some embodiments, the wall thickness of the blade or the shaft is discontinuous, such as due to the presence of an engagement element or a window, respectively, for example. (FIG. 2A.) In other embodiments, the wall thickness of the blade and/or the shaft comprises tapered sections, partially tapered sections, constant thickness sections, discontinuous sections, or combinations thereof. A person of ordinary skill in the art will recognize that additional ranges within the explicit ranges above for the relative wall thicknesses are contemplated and are within the present disclosure.

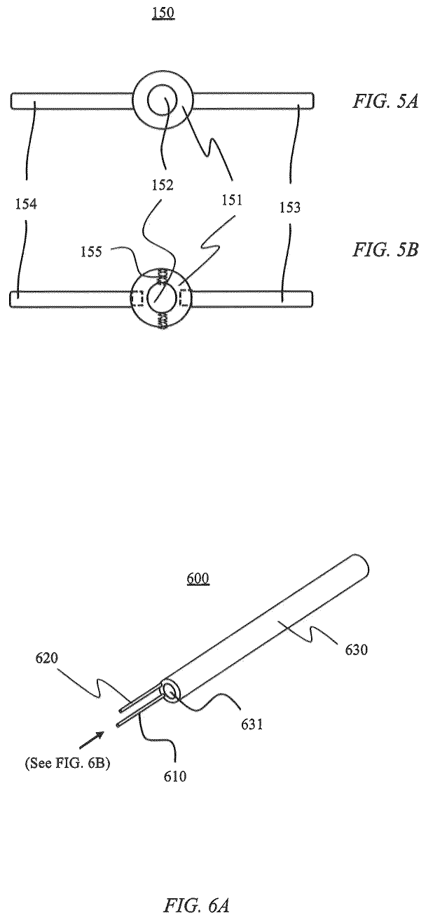

Handle. For convenience and efficiency of operation, the bone harvester 100 can further comprise a handle comprising a handle module 150, which is configured integral to or coupled with proximal end 112 of the shaft 110. The handle module 150 can be mechanically attached to the shaft 110 and one or more rods, wherein the handle module comprises a longitudinal hole extending through the handle module 150 and one or more lateral holes, wherein the shaft 110 extends through the longitudinal hole and each of the one or more rods extends from a lateral hole approximately perpendicular to a longitudinal axis of the shaft. In some embodiments, the handle module 150 is assembled at the proximal end 112 of the shaft 110. (FIGS. 1A-1B). The handle module 150 can comprise a body 151 comprising a through hole 152 configured to couple with the proximal end 112 of the shaft 110 so that the handle module 150 can be assembled at the shaft 110. In some embodiments, the body 151 further comprises at least one rod extending from the body 151 perpendicularly to a longitudinal axis of the shaft 110 (FIG. 1C, FIG. 5A and FIG. 5B). A first rod 153 and a second rod 154 are shown in this example. In some embodiments, the two rods are extending from opposites sides of the body 151 with their longitudinal axes aligned.

In some embodiments, the through hole 152 comprises an inner diameter matching an outer diameter of the proximal end 112 of the shaft 110 so that the through hole 152 can accommodate the proximal end 112 of the shaft 110 stably. In some embodiments, the shaft 110 comprises an opening at the proximal end 112 thereof connecting the lumen 113, and the assembly of the shaft 110 and the handle module 150 does not block the opening. The assembly of the shaft 110 and the handle module 150 can be via a bolt or a screw, which penetrates the body 151 of the handle module 150 through a screw hole 155 thereby mounting the handle module 150 on the shaft 110 (FIG. 5B), or any other convenient fastener. In some embodiments, there are more than one screw holes 155 for better stability.

In some embodiments, handle module 150 and shaft 110 (or a part of the shaft) of the bone harvester can be a unitary structure, meaning the handle module and the shaft (or a part thereof) can manufactured as one structure or joined as an integral structure instead of separate structures.

### Bone Harvesting Guide

In a treatment conducted to facilitate treatment of a cartilage defect according to the present disclosure, more than one bone core might be collected from a defect site of cartilage. After the bone cores are removed, holes will be left at the defect site. For the sake of the efficacy of the treatment, which will be described in more detail below, it is generally desirable to keep holes equidistant from one another to maintain the overall integrity of the structure. To that end, another aspect of the present disclosure provides a bone harvesting guide to facilitate the process of removing multiple bone cores from a selected region.

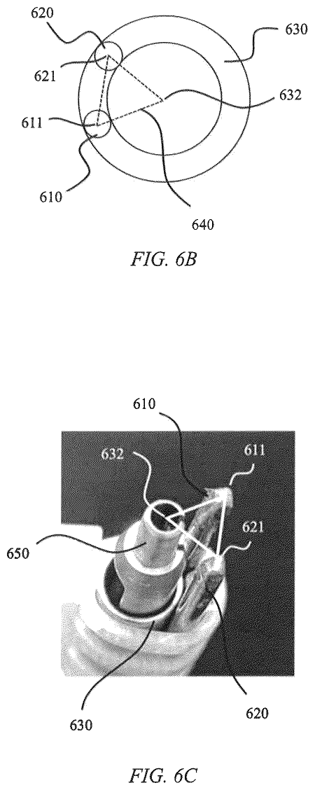

The bone harvesting guide 600 of the present disclosure comprises a first guiding pin 610, a second guiding pin 620, and a tube 630. Tube 630 has a through hole 631 configured to accommodate a bone harvester (FIG. 6A). In this embodiment, first guiding pin 610, second guiding pin 620, and tube 630 are assembled into a fixed configuration whereby the longitudinal axis of first guiding pin 610, a longitudinal axis of second guiding pin 620, and a longitudinal axis of tube 630 are substantially parallel to each other. In some embodiments, as depicted in FIG. 6A, first guiding pin 610 and second guiding pin 620 are attached to an end of tube 630. In other embodiments, first guiding pin 610 and second guiding pin 620 are attached to an outer wall of tube 630.

Moreover, on a cross-section of the bone harvesting guide 600 (See FIG. 6B), a center 611 of first guiding pin 610, a center 621 of second guiding pin 620, and a center 632 of the tube 630 can approximately form vertexes of an equilateral triangle 640. When a bone harvester 650 is assembled within the tube 630, the longitudinal axis of bone harvester 650 is aligned and overlaid with the longitudinal axis of tube 630. Thus, a center of bone harvester 650 on a cross-section is at the same vertex of equilateral triangle 640 as center 632 of tube 630 (FIG. 6C). FIG. 6C also shows center 611 of first guiding pin 610 and center 621 of second guiding pin 620 being the other two vertexes of equilateral triangle 640. This configuration then provides for removal of a bone plug at one of the vertices of the equilateral triangle. If a guiding pin is placed in a previously formed hole or if both guiding pins are placed into two previously formed holes, removal of a bone plug using tube 630 results in the removal of a bone plug at the selected fixed distance of the harvesting guide, and subsequent plug removal can result in removal of bone plugs at the three vertices of the equilateral triangle.

With this design of bone harvester guide 600, the first guiding pin and the second guiding pin are configured to insert into holes from which bone cores were collected so that the next bone core can be collected and leaving a hole that is distanced from other holes at a substantially the same distance. The use of the bone harvesting guide of the present disclosure is described in more details below. Accordingly, in some embodiments, a distal end of the first guiding pin comprises a diameter, which is not greater than a diameter of a bone core to be harvested by the bone harvester. Likewise, a distal end of second guiding pin comprises a diameter, which is not greater than a diameter of a bone core to be harvested by the bone harvester. In certain embodiments, the diameter of the bone core can be as described above.

In some embodiments, a distance between any two of center 632 of the tube (or the center of bone harvester 650), center 611 of first guiding pin 610, and center 621 of second guiding pin 620 is about 4, 4.5, 5, 6, 6.5, 7, 7.5, 8, 8.5, 9 mm, or a range therein. In certain embodiments, the distance can be 4 to 9 mm, 4 to 8.5 mm, 4 to 7.5 mm, 4 to 7 mm, 4 to 6.5 mm, 4 to 6 mm, 4 to 5 mm, 5 to 9 mm, 5 to 8.5 mm, 5 to 7.5 mm, 5 to 7 mm, 5 to 6.5 mm, or 5 to 6 mm. A person of ordinary skill in the art will recognize that additional ranges of center to center distances within the explicit ranges above are contemplated and are within the present disclosure. The size of the bone harvester guide is conducive for use in a less invasive arthroscopic procedure.

Furthermore, while being assembled, a distance (e.g., a shortest distance) between the center of first guiding pin 610 or the center of second guiding pin 620 to the center of the shaft (e.g., a side wall of the blade) is about 3, 3.5, 4, 4.5, 5, 5.5, 6, 6.5, 7, 7.5, 8, 8.5, 9, 9.5, or 10 mm, or a range therein. In certain embodiments, the distance can be 3 to 10 mm, 3 to 9.5 mm, 3 to 9 mm, 3 to 8 mm, 3 to 7 mm, 3 to 6 mm, 3 to 5 mm, 3 to 4 mm, 4 to 8 mm, 4 to 7 mm, 4 to 6 mm, or 4 to 5 mm. In certain embodiments, while being assembled, a distance (e.g., a shortest distance) between a side wall of first guiding pin 610 or a side wall of second guiding pin 620 to a side wall of the distal end of the shaft (e.g., a side wall of the blade) can be about 3, 3.5, 4, 4.5, 5, 5.5, 6, 6.5, 7, 7.5, 8 mm, or a range therein. In certain embodiments, the distance can be 3 to 8 mm, 3 to 7 mm, 3 to 6 mm, 3 to 5 mm, 3 to 4 mm, 4 to 8 mm, 4 to 7 mm, 4 to 6 mm, or 4 to 5 mm. A person of ordinary skill in the art will recognize that additional ranges of pin distances within the explicit ranges above are contemplated and are within the present disclosure.

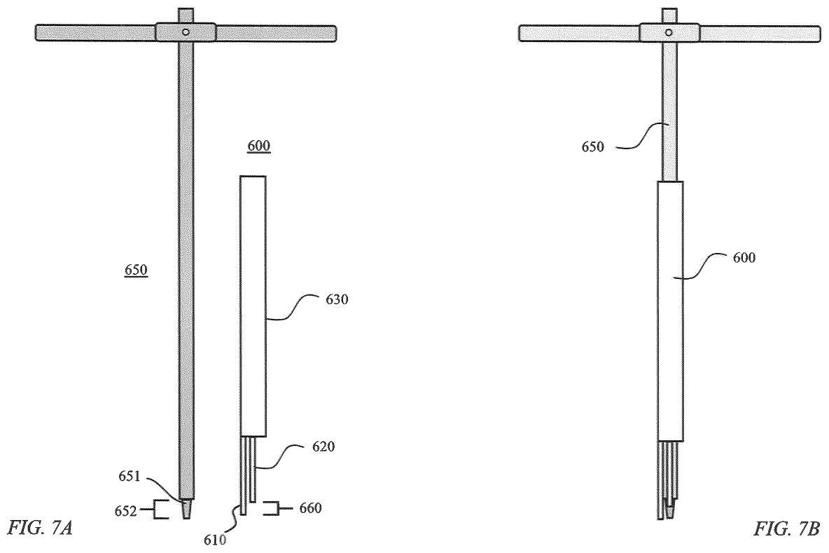

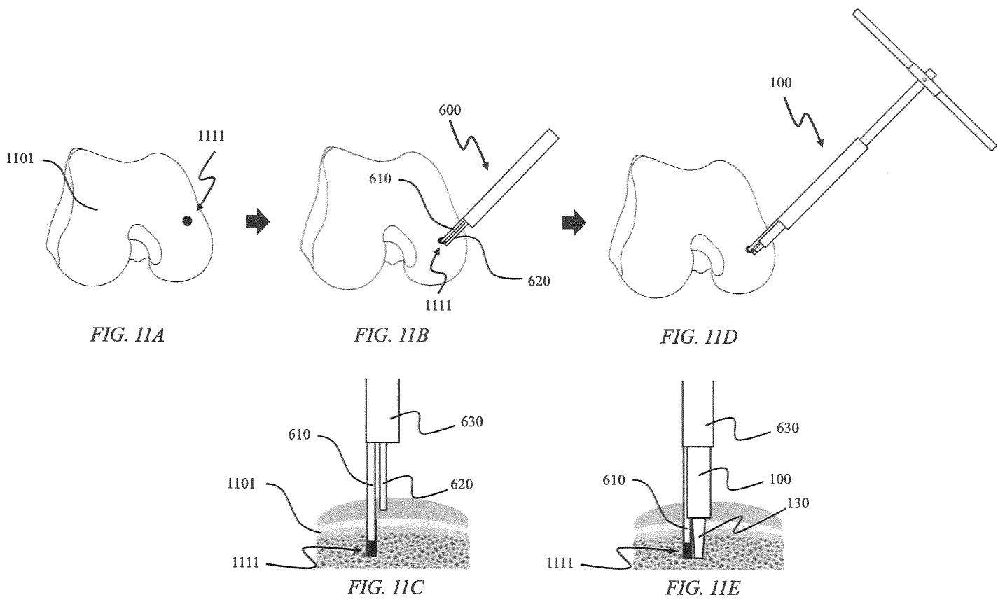

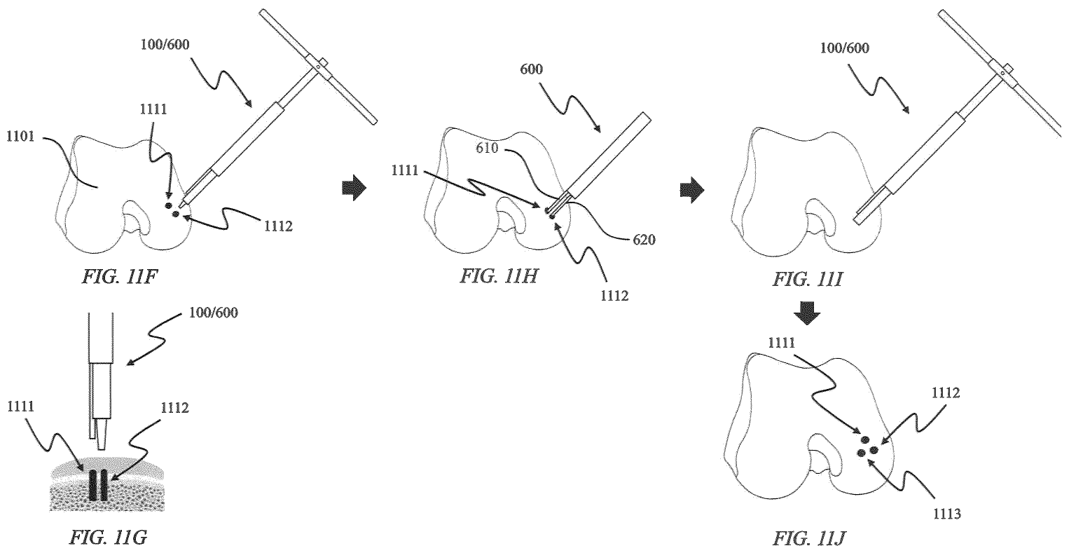

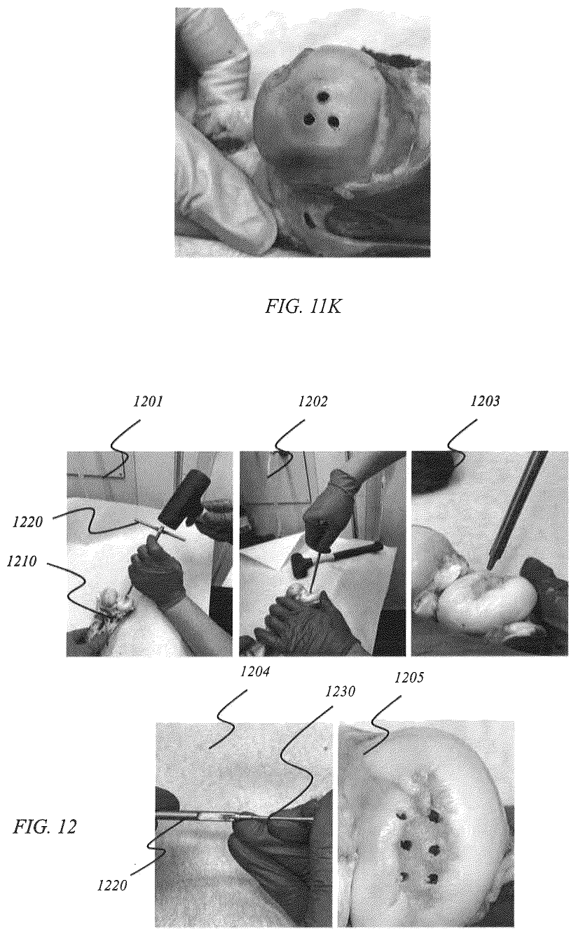

FIG. 7A illustrates a bone harvester 650 and a bone harvesting guide 600 from a side view. As shown in the figures, first guiding pin 610 extends longer by a length 660 than second guiding pin 620 at a distal end of bone harvesting guide 600. Length 660 is greater than zero. In some embodiments, length 660 is about 1 mm to about 12 mm. In other embodiments, length 660 is at least about 1, 0.5, 0.4, 0.3, or 0.25 mm and no more than 2, 3, 4, 5, 6, 7, 8, 9, 1, 11 mm. A person of ordinary skill in the art will recognize that additional ranges of length are within the present disclosure. In some embodiments, length 660 is not more length 652 of tip 651 of bone harvester 650, which is configured to insert into a bone. As described above, in some embodiments tip 651 of bone harvester 650 is a blade of bone harvester 650. Therefore, length 660 is smaller than a length of a bone core to be collected. This is because, when bone harvesting guide 600 is used together with bone harvester 650/100 for collecting a second bone core (for example, see FIGS. 11B to 11E), first guiding pin 610 is be inserted into first hole 1111 from which a first bone core was collected. Referring to FIG. 11B, at this stage there is only one hole in bone 1101 for inserting first guiding pin 610. If the two guiding pins are equal in length (e.g., length 660 equals zero), the first guiding pin would not be able to extend into first hole 1111 due to second guiding pin 620 contacting the surface of bone 1101. Providing bone harvesting guide 600 with length 660 assures that when second guiding pin 620 contacts the surface of bone 1101 and cannot extend further, first guiding pin 610 extends into first hole 1111 to position bone harvester 650/100 for obtaining the second bone core. Nevertheless, length 660 should not be too short. Referring to FIGS. 11H-11I, when bone harvesting guide 600 is used together with bone harvester 650/100 for collecting a third bone core, first guiding pin 610 and second guiding pin 620 are inserted into first hole 1111 and second hole 1112, respectively, for positioning the bone harvester. In other words, second guiding pin 620 should be short enough so that it won't interfere with the collection of a second bone core but long enough to assist the collection of a third bone core.

In certain embodiments, the length 660 can be about half a length of a bone core to be collected. The difference between the length 660 and the length 652 can be observed more clearly while bone harvester 650 and bone harvesting guide 600 are assembled (FIG. 7B). In alternative embodiments, one or both pins can have lengths that are adjustable to allow for pins to insert into previously drilled holes or not while maintaining the axis of bone harvester 650 within bone harvester guide 600 roughly perpendicular to the bone surface for drilling.

In some embodiments, length 660 corresponding to different lengths of guiding pins 610, 620 can be about 2, 2.5, 3, 3.5, 4, 4.5, 5, 5.5, 6, 6.5, 7, 7.5, 8, 9, 10, 11, 12 mm, or a range therein. In certain embodiments, the distance can be 2 to 12 mm, 2 to 11 mm, 2 to 10 mm, 2 to 9 mm, 2 to 8 mm, 2 to 7 mm, 2 to 6 mm, 2 to 5 mm, 2 to 4, 2 to 3 mm, 3 to 12 mm, 3 to 11 mm, 3 to 10 mm, 3 to 9 mm, 3 to 8 mm, 3 to 7 mm, 3 to 6 mm, 3 to 5 mm, 3 to 4 mm, 4 to 12 mm, 4 to 11 mm, 4 to 10 mm, 4 to 9 mm, 4 to 8 mm, 4 to 7 mm, 4 to 6 mm, 4 to 5 mm, 5 to 9 mm, 5 to 8 mm, 5 to 7 mm, 5 to 6 mm, 6 to 12 mm, 6 to 8 mm, 7 to 12 mm, or 7 to 10 mm. A person of ordinary skill in the art will recognize that additional ranges of length 660 within the explicit ranges above are contemplated and are within the present disclosure.

In some embodiments, tube 630 has a length from about 10 mm to about 150 mm, from about 20 mm to about 150 mm, from about 20 mm to about 100 mm, or from about 50 mm to about 100 mm. In some embodiments, the first guiding pin and the second guiding pin independently have a diameter that is approximately the same as or smaller than the diameter of a harvested bone core. In certain embodiments, first guiding pin 610 and second guiding pin 620 independently have a diameter from about 0.25 mm to about 5 mm.

### Pushing Tool

After a bone core is collected and retrieved within a bone harvester, it can be desirable to efficiently remove the collected bone core from the bone harvester without causing substantial damage to the bone core. Therefore, another aspect of the present disclosure provides a pushing tool that is configured to operate collaboratively with the bone harvester of the present disclosure. Alternative embodiments of a pushing tool are described in the following.

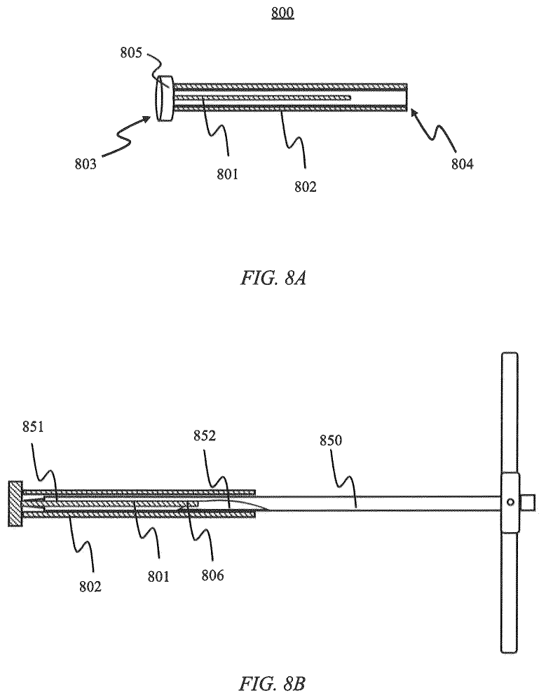

In some embodiments, the collected bone core can be removed from a window of the bone harvester. FIG. 8A illustrates a cross-sectional view of a pushing tool 800 according to an embodiment of the present disclosure. The pushing tool 800 comprises a pushing rod 801, which can have any suitable construction. In some embodiments, the pushing rod 801 further comprises a blind tube 802 having a closed end 803 and an open end 804. In such embodiments, the pushing rod 801 is positioned inside the inner space with an end thereof mounted at the closed end 803 of the blind tube 802. In some embodiments, the blind tube 802 defines an inner space having the same cross-sectional area along a longitudinal axis from the closed end 803 to the open end 804. In some embodiments, the closed end 803 of the pushing tool 800 further comprises a head 805 wherein the pushing rod 801 is mounted.

See FIG. 8B. In some embodiments, the pushing tube 802 defines an inner space, which is configured to accommodate a distal end 851 of a shaft 850 of a bone harvester according to the present disclosure. In certain embodiments, the cross-sectional area of the inner space of blind tube 802 is substantially the same as a cross-sectional area of distal end 851 of shaft 850. In some embodiments, when blind tube 802 is mounted on shaft 850, distal end 851 of shaft 850 can be pushed at most against an inner surface of closed end 803 (e.g., head 805) of blind tube 802, which provides sufficient range of motion to push the bone plug through a window.

Pushing rod 801 is configured to push a collected bone core out of the lumen of shaft 850, for example, from a window 852 of the bone harvester. In some embodiments, pushing rod 801 has a shape matching the lumen defined by shaft 850. The term “matching the lumen” does not mean that the shape of pushing rod 801 should be the same as the shape of the lumen but means that the shape of pushing rod 801 should allow it to be pushed into the lumen and preferably deep enough to push a bone core out of the lumen. In some embodiments, pushing rod 801 has a length that, when blind tube 802 is assembled with shaft 850, a free end 806 (an end that is not mounted at blind tube 802) of pushing rod 801 is visible from window 852 of shaft 850.

In some embodiments, pushing rod 801 has a width matching a dimension the lumen defined by shaft 850. The term “matching a dimension of the lumen” does not mean that the width of pushing rod 801 is the same as the dimension of the lumen but means the width of pushing rod 801 should allow it to be pushed into the lumen while is wide enough to efficiently push a collected bone core out of the lumen. In certain embodiments, pushing rod 801 has an outer diameter, which is shorter or substantially the same length as an inner diameter of shaft 850.

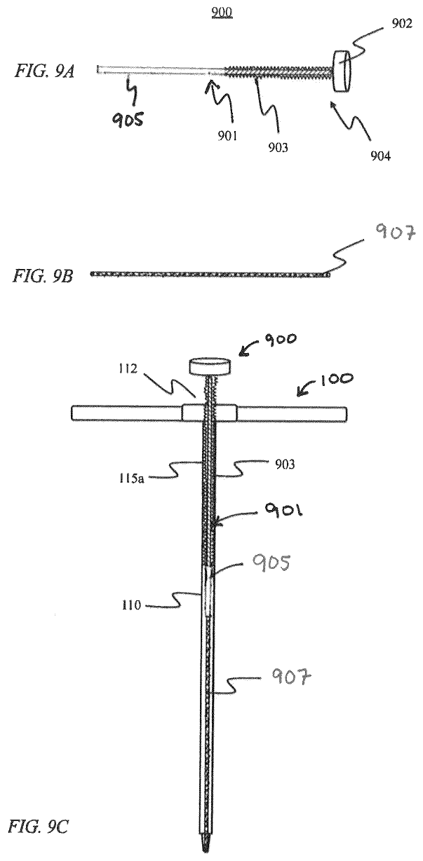

In a different embodiment of a pushing tool 900 according to the present disclosure (FIG. 9A), pushing tool 900 comprises a pushing rod 901 mounted at a head 902. Pushing tool 900 is configured to push a collected bone core out from a proximal end of a bone harvester. Pushing tool 900 of this embodiment is particularly configured to be used with, for example bone harvester 100a as shown in FIG. 1C. As illustrated in FIG. 1C, shaft 110 of the bone harvester 100a comprises a first portion and a second portion, wherein the first portion is positioned proximal to distal end 111 thereof and comprises a smooth inner surface 114; and wherein the second portion is positioned proximal to proximal end 112 thereof and comprises a threaded inner surface 115.

The threaded inner surface 115 comprises a first thread 115a, and the pushing rod 901 comprises a second thread 903 that matches the first thread 115a. The second thread 903 can be positioned immediately distal to proximal end 904 of pushing rod 901. In some embodiments, pushing rod 901 further comprises a distal portion 905 that is not covered by the second thread as shown in FIG. 9A.

FIG. 9C illustrates the assembly of the pushing tool 900 with the bone harvester 100. As illustrated in the figure, the pushing tool 900 inserts into lumen of shaft 110 from the opening at the proximal end 112 thereof. While inserting, the second thread 903 screws with the first thread 115a thereby driving/screwing pushing rod 901 into the lumen and eventually pushing a collected bone core out of the lumen. In some embodiments, the pushing rod 901 is sufficiently long to reach and push a collected bone core out of the lumen. In other embodiments, pushing tool 900 further comprises a leading rod 907 (FIG. 9B). Leading rod 907 is configured to be put into the lumen before screwing the pushing rod 901 so that while screwing, pushing rod 901 pushes leading rod 907 forward until the leading rod 901 push a collected bone core out of the lumen.

In some embodiments, leading rod 907 has a shape matching the lumen defined by the shaft. Thus, if the lumen defined by the shaft has a non-cylindrical shape, such as created with a non-circular cross section, leading rod 907 can insert into the lumen due to a smaller diameter or a matching shape. The term “matching the lumen” does not mean that the shape of leading rod 907 should necessarily be the same as the shape of the lumen but means that the shape of leading rod 907 should allow it to be pushed into the lumen deep enough to push a bone core out of the lumen. In alternative embodiments, pushing rod 901 or an appropriate distal portion thereof has an appropriate shape and length to push a bone core out of the lumen. In certain embodiments, leading rod 907 comprises a diameter substantially the same with a diameter of the pushing rod 901.

### Kits.

Another aspect of the present disclosure provides a kit for harvesting a bone core. In some embodiments, the kit can comprise a bone harvester of the present disclosure and a pushing tool of the present disclosure. In some embodiments, the kit comprises the bone harvesting guide of the present disclosure and the bone harvester of the present disclosure. In some embodiments, the kit comprises the bone harvester of the present disclosure, the bone harvesting guide of the present disclosure, and the pushing tool of the present disclosure.

### Methods for Processing a Bone

Another aspect of the present disclosure provides a method for processing a bone. In some embodiments, the method is performed to collect a bone core from a bone. In certain embodiments, the method is performed to collect more than one bone cores from a bone. In some embodiments, the method is performed to create a hole on a bone, which can result from removal of a bone core.

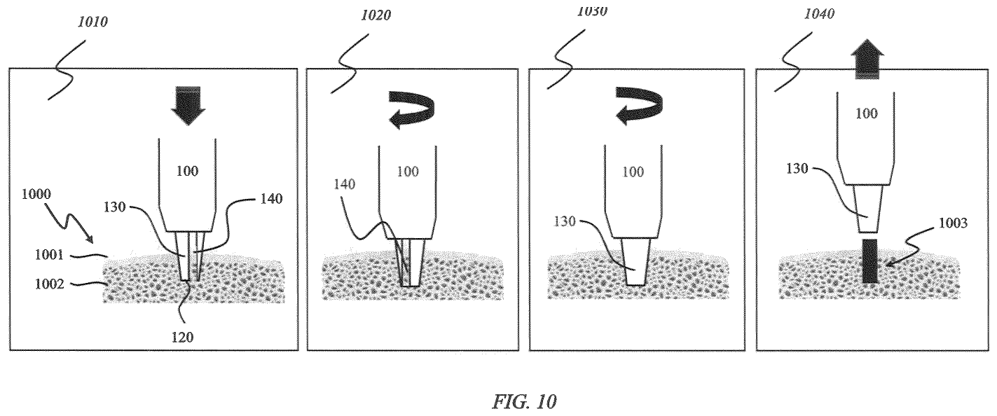

Referring to FIG. 10, the method of the present disclosure comprises inserting a bone harvester 100 of the present disclosure into a bone 1000, which may involve placement of a portion of the engagement element (i.e., open slot 140) of the bone harvester 100 within the bone 1000 (See Box 1010). Other embodiments of a bone harvester described herein similarly interface with bone 1000 to form a corresponding bone plug. As depicted in FIG. 10, blade 130 of the bone harvester 100 is inserted into the bone 1000 by cutting through bone 1000 with cutting edge 120. Some bone tissues are retained within the blade and can be seen from the open slot 140 in this figure.

In some embodiments, inserting bone harvester 100 comprises inserting the cutting edge 120 thereof into cartilage 1001 (e.g., an articular cartilage) of the bone 1000, specifically by contacting the cutting edge 120 of the bone harvester 100 with a surface of the cartilage 1001. In certain embodiments, inserting the bone harvester 100 comprises inserting the cutting edge 120 thereof into a subchondral bone 1002 of the bone 1000. In certain embodiments, inserting the bone harvester 100 comprises applying a compressive force to the proximal end of the shaft. Applying the compressive force can comprise hitting the bone harvester 100 by bare hand or by impacting the proximal end of the shaft with a hammer or mallet.

It can be desirable to insert bone harvester 100 into bone 1000 to have step 132 up against a surface of cartilage 1001 or otherwise against the surface of bone 1000. In appropriate embodiments, for the open slot 140 to efficiently perform its function of breaking/snapping a root of the bone core from the bone 1000, at least 10%, 20%, 30%, 40%, 50%, 60%, 70%, 80%, 90%, 95%, or a range therein of the open slot by its length is within the bone 1000. In certain embodiments, the entire open slot 140 is within the bone 1000. In some embodiments, 10 to 100%, 20 to 100%, 30 to 100%, 40 to 100%, 50 to 100%, 60 to 100%, 70 to 100%, 80 to 100%, 90 to 100%, 95 to 100%, 10 to 95%, 20 to 95%, 30 to 95%, 40 to 95%, 50 to 95%, 60 to 95%, 70 to 95%, 80 to 95%, 90 to 95%, 20 to 90%, 30 to 80%, 40 to 70%, or 50 to 60% of the open slot by its length is within the bone 1000. In further embodiments of bone harvester 100, at least 50%, 60%, 70%, 80%, 90% or 100% (and corresponding ranges 50-100%, 60-100%, 70-90%, etc.) of length of the blade of bone harvester 100 up to step 132 is inserted into the bone. A person of ordinary skill in the art will recognize that additional ranges of insertion within the explicit ranges above are contemplated and are within the present disclosure.

Then, the method comprises rotating the bone harvester 100 relative to the bone 1000 while the portion of the open slot 140 or the blade 130 of bone harvester 100 maintains within the bone 1000 (Box 1020). As described above, while rotating the bone harvester 100, the cutting edge 120 cuts through the bone, and an edge of the open slot 140 applies torque to snap the root of the retained bone tissue (a bone core) from the bone 1000. The rotation can continue until the bone core is separate from the bone 1000 (Box 1030). Once the bone core is separated, bone harvester may freely rotate. Next, the method comprises withdrawing the bone harvester 100 from the bone 1000 thereby obtaining a harvested bone core within the through-bore (now shown in this figure) of the blade 130 (Box 1040). A hole 1003 remains in the bone where the bone plug is removed. Rotation of bone harvester 100 can be performed manually by rotating handle, or the bone harvester can engage a drill or other motorized tool to rotate the bone harvester with power drive.

In some embodiments, rotating the bone harvester comprises rotating the bone harvester for at least about 5, 10, 15, 20, 25, 30, 35, 40, 45, 50, 55, 60, 65, 70, 75, 80, 85, 90, 95, 100, 120, 150, 200, 250, 300, 360 degrees, or a range therein. The rotating can be performed by operating a handle module of the bone harvester as described above. While it is not limited, the rotating can be performed in a clockwise direction or an anti-clockwise direction or alternatingly in a first direction followed by the opposite direction.

In some embodiments, withdrawing the bone harvester 100 comprises withdrawing the bone harvester 100 out of the bone 1000 and leaving a hole 1003 at the bone (Box 1040). The hole 1003 has a shape corresponding to the harvested bone core. Dimensions of a harvested bone core can be as described above.

In some embodiments, the method further comprises removing the harvested bone core out of the bone harvester. In specific embodiments, removing the harvested bone core comprises using the pushing tool of the present disclosure as described above.

In some embodiments, the method comprises harvesting more than one bone core from the same bone and leaving more than one hole on the bone. In order to have each hole is substantially equidistant from each other, the bone harvesting guide of the present disclosure can be used. In some embodiments, 3, 4, 5, 6, 7, 8, 9, or more bone cores are harvested from the same bone and corresponding 3, 4, 5, 6, 7, 8, 9, or more holes are left on the bone. In some embodiments, any distances between any two of the 3, 4, 5, 6, 7, 8, 9, or more holes can be substantially the same. In some embodiments, the distance refers to a distance between centers of any two of the 3, 4, 5, 6, 7, 8, 9, or more holes. In some embodiments, the distance refers to a shortest straight distance between edges of two of the 3, 4, 5, 6, 7, 8, 9, or more holes. While in general, various patterns can be formed with the bone harvesting guide, one convenient pattern has 7 holes with one hole at the center and the other six holes at the vertices of a regular hexagon. This pattern is formed by placing a center hole and rotating the bone harvesting guide around the center hole to form equidistant holes that correspond to the vertices of the hexagon, with 60 degrees being the angle of the equilateral triangle.

Referring to FIG. 11A, a first hole 1111 is inserted into the bone 1101 after a first bone core is harvested. To harvest a second bone core, a bone harvesting guide 600 of the present is used. As illustrated in FIG. 11B, the method of the present disclosure comprises inserting the first guiding pin 610 into the first hole 1111. FIG. 11C illustrates a cross-sectional view showing that the first guiding pin 610 is inside the first hole 1111. As explained above, it is noted that the first guiding pin 610 extends further than the second guiding pin 620 so that while the first guiding pin 610 is inside the first hole 1111, the second guiding pin 620 barely touches the surface of the bone 1101.

Then, the method comprises assembling the bone harvester 100 with the tube 630 of the bone harvesting guide 600 on the bone 1101 (FIG. 11D). The bone harvester 100 is then pushed to have its blade 130 cut into the bone 1101 while the first guiding pin 610 is maintained inside the first hole 1111 (FIG. 11E). A second bone core is then harvested following the same steps as described above for harvesting the first bone core, leaving a second hole 1112 on the bone 1101 after the bone harvester 100 is withdrawn (FIG. 11F and FIG. 11G).

Next, in order to harvest a third bone core, the bone harvesting guide 600 is again inserted into the bone. This time, the first guiding pin 610 can be inserted into either the first hole 1111 or the second hole 1112, and the second guiding pin 620 can be inserted into the other one. In some embodiments, the first guiding pin 610 is inserted into the first hole 1111, and the second guiding pin 620 is inserted into the second hole 1112 (FIG. 11H). In some embodiments, the first guiding pin 610 is inserted into the second hole 1112, and the second guiding pin 620 is inserted into the first hole 1111. Then, following the same steps as described above, the bone harvester 100 is assembled with the bone harvesting guide 600 and is pushed to cut into the bone to harvest a third bone core (FIG. 11I). A third hole 1113 is left on the bone 1101 after the bone harvester 100 is withdrawn (FIG. 11J). The first hole 1111, the second hole 1112, and the third hole 1113 are substantially equidistant from one another (FIG. 11K).

In some embodiments, the first hole, the second hole, and the third hole are located from one another at a distance of about 4, 4.5, 5, 6, 6.5, 7, 7.5, 8, 8.5, 9 mm, or a range therein. The distance is defined as a distance between the center of the first hole, the center of the second hole, and the center of the third hole. In certain embodiments, the distance can be 4 to 9 mm, 4 to 8.5 mm, 4 to 7.5 mm, 4 to 7 mm, 4 to 6.5 mm, 4 to 6 mm, 4 to 5 mm, 5 to 9 mm, 5 to 8.5 mm, 5 to 7.5 mm, 5 to 7 mm, 5 to 6.5 mm, or 5 to 6 mm. A person of ordinary skill in the art will recognize that additional ranges of distances within the explicit ranges above are contemplated and are within the present disclosure.

In some embodiments, the bone is of a patient having osteoarthritis or cartilage injury. In some embodiments, the bone is of a patient having cartilage defects. In certain embodiments, the bone has a partial thickness defect or full thickness defect.

The bone harvester, however, is not necessary to be used together with the bone harvesting guide. FIG. 12 show photos of an actual experiment of performing the method of processing a bone using a bone harvester according to the present disclosure without using the bone harvesting guide. The experiment used a pork knee joint 1210 for bone core harvest. A bone harvester 1220 of the present disclosure was pushed by a hammer and cut into the bone (Step 1201). Then, the bone harvester was rotated (Step 1202) relative to the bone and withdrawn (Step 1203). Then, the collected bone core was removed out of the bone harvester 1220 using a pushing tool 1230. Then, five more bone cores were collected using the bone harvester 1220 (not shown). Six bone cores were collected, and six holes were left on the joint 1210 (Step 1205).

FIG. 13A and FIG. 13B show photos of a bone core harvested by using the bone harvester of the present disclosure. The bone core has a distorted cylindrical body 1301 having a non-circular cross section perpendicular to the cylindrical axis corresponding to an accretion to or removal from the corresponding circular form. The distorted cylindrical body can comprise a ridge 1302 extending from a side of the distorted cylindrical body. The ridge 1302 is resulted from the open slot of the bone harvester. In other embodiments, the distorted cylindrical body can comprise a channel protruding into a side of the distorted cylindrical body, or a flat portion along a side of the distorted cylindrical body, or a combination thereof as a result of other types of engagement elements described herein. In some embodiments, the bone core has a layer of cartilage tissue 1303 and a layer of subchondral tissue 1304. Ranges of dimensions of the bone plugs are described in detail above and reproduced in the following.

In some embodiments, the cylindrical body has a diameter of about 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12 mm, or a range therein. In some embodiments, the diameter is about 0.75 to about 12 mm, 1 to 12 mm, 1.2 to 12 mm, 1.5 to 12 mm, 2 to 12 mm, 3 to 12 mm, 4 to 12 mm, 5 to 12 mm, 6 to 12 mm, 7 to 12 mm, 8 to 12 mm, 9 to 12 mm, 10 to 12 mm, 11 to 12 mm, 1 to 9 mm, 1.2 to 9 mm, 1.5 to 9 mm, 2 to 9 mm, 3 to 9 mm, 4 to 9 mm, 5 to 9 mm, 6 to 9 mm, 7 to 9 mm, 8 to 9 mm, 1 to 6 mm, 1.2 to 6 mm, 1.5 to 6 mm, 2 to 6 mm, 3 to 6 mm, 4 to 6 mm, 5 to 6 mm, 1 to 1.2 mm, 1 to 1.5 mm, 1 to 2 mm, 1 to 2.5 mm, 1 to 3 mm, 1 to 4 mm, 1 to 5 mm, 1.2 to 1.5 mm, 1.2 to 2 mm, 1.2 to 2.5 mm, 1.2 to 3 mm, 1.2 to 4 mm, 1.2 to 5 mm, 1.5 to 2 mm, 1.5 to 2.5 mm, 1.5 to 3 mm, 1.5 to 4 mm, or 1.5 to 5 mm.

In some embodiments, the cylindrical body has a length of about 2, 2.5, 3, 3.5, 4, 4.5, 5, 5.5, 6, 6.5, 7, 7.5, 8, 9, 10, 11, 12 mm, or a range therein. In some embodiments, the bone core can be collected by using the bone harvester of the present disclosure has a length of about 2 to 12 mm, 3 to 12 mm, 4 to 12 mm, 5 to 12 mm, 6 to 12 mm, 7 to 12 mm, 8 to 12 mm, 9 to 12 mm, 10 to 12 mm, 11 to 12 mm, 3 to 11 mm, 4 to 11 mm, 5 to 11 mm, 6 to 11 mm, 7 to 11 mm, 8 to 11 mm, 9 to 11 mm, 10 to 11 mm, 4 to 9 mm, 5 to 9 mm, 6 to 9 mm, 7 to 9 mm, 8 to 9 mm, 2 to 4 mm, 3 to 4 mm, 4 to 5 mm, 4 to 6 mm 4 to 7 mm, 4 to 8 mm, 5 to 6 mm 5 to 7 mm, or 5 to 8 mm.

In some embodiments, the ridge has a length of about 2, 2.5, 3, 3.5, 4, 4.5, 5, 5.5, 6, 6.5, 7, 7.5, 8, 9, 10, 11, 12 mm, or a range therein. In some embodiments, the bone core can be collected by using the bone harvester of the present disclosure has a length of about 2 to 12 mm, 3 to 12 mm, 4 to 12 mm, 5 to 12 mm, 6 to 12 mm, 7 to 12 mm, 8 to 12 mm, 9 to 12 mm, 10 to 12 mm, 11 to 12 mm, 3 to 11 mm, 4 to 11 mm, 5 to 11 mm, 6 to 11 mm, 7 to 11 mm, 8 to 11 mm, 9 to 11 mm, 10 to 11 mm, 4 to 9 mm, 5 to 9 mm, 6 to 9 mm, 7 to 9 mm, 8 to 9 mm, 2 to 4 mm, 3 to 4 mm, 4 to 5 mm, 4 to 6 mm 4 to 7 mm, 4 to 8 mm, 5 to 6 mm 5 to 7 mm, or 5 to 8 mm.

In some embodiments, the ridge has a height of 0.1, 0.2, 0.3, 0.4, 0.5, 0.6, 0.7 mm, or a range therein. In some embodiments, the height is about 0.1 to 0.7 mm, 0.1 to 0.6 mm, 0.1 to 0.5 mm, 0.1 to 0.4 mm, 0.1 to 0.3 mm, 0.1 to 0.2 mm, 0.2 to 0.7 mm, 0.2 to 0.6 mm, 0.2 to 0.5 mm, 0.2 to 0.4 mm, 0.2 to 0.3, 0.3 to 0.7 mm, 0.3 to 0.6 mm, 0.3 to 0.5 mm, 0.3 to 0.4 mm, 0.4 to 0.7 mm, 0.4 to 0.6 mm, 0.4 to 0.5 mm, 0.5 to 0.7 mm, 0.5 to 0.6 mm.

### Material and Construction

All or any of the structural components of the tools, including the bone harvester, handle module, bone harvester guide, and/or pushing tool can be made of metal, alloy, plastic, combinations thereof, or any suitable materials depending on the preference or actual needs. In some embodiments, the bone harvester can be made of metal, the handle module can be made of plastic. In some embodiments, the shaft of the bone harvest and the handle module can be made of plastic, and the blade of the bone harvest can be made of metal. The plastic can include but not limited to nylon, acrylonitrile butadiene styrene (ABS), polypropylene (PP), polystyrene (PS), polyoxymethylene (POM), polyetheretherketone (PEEK), polytetrafluoroethylene (PTFE), high-density polyethylene (HDPE), polycarbonate, polymethyl methacrylate (PMMA), a combination thereof. The metal can include but not limited to 300 series stainless steel, 400 series stainless steel, 600 series stainless steel, titanium, or a combination thereof.

It can be desirable for the tool elements to be sterilizable without damage, such as using an autoclave, UV radiation, ethylene oxide, other penetrating radiation or the like. Tool elements can be machined using conventional tool manufacturing procedures and equipment. The tools can be distributed using approved regulatory instructions using conventional distribution channels for medical devices.

### Further Inventive Concepts

1. A bone harvester for harvesting a bone core, comprising a shaft having a lumen, a distal end with a blade having a distal cutting edge, a proximal end, and an engagement element that locks the relative rotation of the bone harvester and the bone core.

2. The bone harvester of inventive concept 1, wherein the engagement element comprises an open slot, a notch, a flat face, an internal slot substantially parallel to a longitudinal axis of the shaft, a collar, a plurality thereof, or a combination thereof.

3. The bone harvester of inventive concept 1 or 2, wherein the engagement element is positioned at the blade.

4. The bone harvester of any one of inventive concepts 1-3, wherein the engagement element comprises the open slot, and the open slot has a side opening through the blade that extends from the distal cutting edge toward the proximal end of the shaft.

5. The bone harvester of inventive concept 4, wherein the open slot does not comprise a cutting edge.

6. The bone harvester of inventive concept 4, wherein the open slot comprises edges, which are parallel to a longitudinal axis of the shaft.

7. The bone harvester of inventive concept 4, wherein a length of the open slot is at least 20% of a length of the bone core.

8. The bone harvester of inventive concept 7, wherein the length of the open slot is at least 50% of the length of the bone core.

9. The bone harvester of inventive concept 4, wherein a width of the open slot is from 5% to 10% of the circumference of the bone core.

10. The bone harvester of inventive concept 4, wherein the open slot has a width from about 0.3 mm to about 7 mm.

11. The bone harvester of inventive concept 1, wherein the engagement element comprises a first open slot and a second open slot.

12. The bone harvester of inventive concept 2, wherein the engagement element comprises the notch, and the notch comprises a protrusion into the lumen of the shaft.

13. The bone harvester of inventive concept 12, wherein the notch has a triangular cross-sectional shape.

14. The bone harvester of inventive concept 13, wherein the notch has a radial extent of at least about 2 mm.

15. The bone harvester of inventive concept 2, wherein the engagement element comprises the internal slot, and the internal slot comprises a channel in a side portion of an inner circumference of the blade that extends from the distal cutting edge toward the proximal end of the shaft.

16. The bone harvest of inventive concept 15, wherein the channel has a radial indent of at least about 2 mm.

17. The bone harvester of inventive concept 2, wherein the engagement element comprises the flat face, and the flat face protrudes into the lumen of the shaft and extends from the distal cutting edge toward the proximal end of the shaft.

18. The bone harvester of inventive concept 17, wherein the engagement element comprises a plurality of the flat faces.

19. The bone harvester of inventive concept 17 or 18, wherein the flat face has a dimension of at least about 2 mm.

20. The bone harvester of any one of inventive concepts 1-19, wherein the length of the bone core is about 4 to 10 mm.

21. The bone harvester of any one of inventive concepts 1-20, wherein the bone core has a diameter from about 2.5 to about 8 mm.

22. The bone harvester of any one of inventive concepts 1-20, wherein the diameter of the bone core is about 1 to 5 mm.

23. The bone harvester of any one of inventive concepts 1-22, wherein the blade has a circumferential shape and has a longitudinal through-bore connecting to the lumen of the shaft.

24. The bone harvester of inventive concept 23, wherein the longitudinal through-bore comprises a first inner diameter, and the lumen of the shaft comprises a second inner diameter, and the first inner diameter is substantially the same as the second inner diameter.

25. The bone harvester of inventive concept 24, wherein a cross-section of the longitudinal through-bore is generally circular.

26. The bone harvester of inventive concept 23, wherein the blade is tapered with a shorter outer diameter at a distal end thereof where the cutting edge is positioned.

27. The bone harvester of any one of inventive concepts 1-26, wherein the blade has an outer diameter at a distal end thereof in a range from about 2 mm to about 22 mm.

28. The bone harvester of inventive concepts 1-27, wherein the blade has a tapered outer diameter along a direction of an axis of the shaft toward the distal end.

29. The bone harvester of inventive concept 28, wherein the blade has an outer diameter that tapers by an amount from about 3 mm to about 5 mm, such that the proximal diameter is larger than the distal diameter.

30. The bone harvester of any one of inventive concepts 1-29, wherein the blade has a tapered wall thickness.

31. The bone harvester of inventive concept 30, wherein the blade has a wall thickness that tapers from about 0.4 mm to about 0.8 mm.

32. The bone harvester of any one of inventive concepts 1-31, wherein the shaft comprises a step adjacent to the blade, wherein the step is formed by a change in outer diameter of the shaft.

33. The bone harvester of inventive concept 32, wherein a diameter of the shaft proximal to the step has a diameter at least 1 mm larger than a diameter of the shaft distal to the step.

34. The bone harvester of inventive concept 32 or 33, wherein the step has a position to form a bone core having a length from about 2 mm to about 12 mm.

35. The bone harvester of any one of inventive concepts 1-34, wherein the shaft further comprises a window proximal to the blade, wherein the window comprises an opening on a side portion of the shaft to expose a portion of the lumen.

36. The bone harvester of inventive concept 35, wherein the window is at a position proximal to the distal end of the shaft.

37. The bone harvester of inventive concept 35, wherein a distal portion of the opening of the window is from about 5 mm to about 15 mm from a proximal portion of the blade.

38. The bone harvester of inventive concept 35, wherein the opening of the window has a length of about 5 mm to about 30 mm, and wherein the opening has a width that is from about 50% to about 100% of the outer diameter of the shaft.

39. The bone harvester of any one of inventive concepts 1-38, wherein a portion of the shaft is a hollow cylinder.

40. The bone harvester of inventive concept 39, wherein the portion of the shaft being a hollow cylinder comprises the lumen.

41. The bone harvester of any one of inventive concepts 1-40, wherein the lumen extends from the distal end to the proximal end of the shaft.

42. The bone harvester of any one of inventive concepts 1-41, wherein the lumen comprises a smooth wall surface, and the shaft comprises a smooth outer surface.

43. The bone harvester of inventive concept 42, wherein the shaft further comprises an inner surface having a thread.

44. The bone harvester of inventive concept 43, wherein the shaft comprises a first portion and a second portion, wherein the first portion comprises a smooth inner surface, and the second portion comprises the threaded inner surface.

45. The bone harvester of inventive concept 44, wherein the first portion of the shaft is positioned proximal to the distal end thereof.

46. The bone harvester of inventive concept 44, wherein the second portion of the shaft is positioned proximal to the proximal end thereof.

47. The bone harvester of any one of inventive concepts 1-46, wherein the shaft has a length from about 100 mm to about 150 mm, and wherein the blade has a length from about 2 mm to about 15 mm.

48. The bone harvester of inventive concept 1, wherein the shaft has a same cross-sectional area along a longitudinal axis thereof.

49. The bone harvester of inventive concept 1, wherein the shaft has a same inner diameter along a longitudinal axis thereof.

50. The bone harvester of any one of inventive concepts 1-49, further comprising a handle permanently secured to the shaft and configured such that manual rotation of the handle provides for rotation of the shaft around its longitudinal axis.

51. The bone harvester of any one of inventive concepts 1-49, further comprising a handle comprising a cavity, wherein the shaft comprises a protrusion at or near its distal end configured to engage the cavity of the handle such that rotation of the handle provides rotation of the shaft around its axis.

52. The bone harvester of any one of inventive concepts 1-49, further comprising a handle comprising a handle module, wherein the handle module is mechanically attached to the shaft and one or more rods, wherein the handle module comprises a longitudinal hole extending through the handle module and one or more lateral holes, wherein the shaft extends through the longitudinal hole and each of the one or more rods extends from a lateral hole approximately perpendicular to a longitudinal axis of the shaft.

53. The bone harvester of inventive concept 52, wherein the handle module is assembled to the proximal end of the shaft via a bolt or a screw.

54. A bone harvesting guide, comprising:
a first guiding pin;
a second guiding pin; and
a tube;
wherein the first guiding pin, the second guiding pin, and the tube are assembled into a connected assembly whereby a longitudinal axis of the first guiding pin, a longitudinal axis of the second guiding pin, and a longitudinal axis of the tube are substantially parallel to each other; and
wherein, in a cross-section of the bone harvesting guide, a center of the first guiding pin, a center of the second guiding pin, and a center of the tube form vertexes of an equilateral triangle.

55. The bone harvesting guide of inventive concept 54, wherein the tube is configured to accommodate a bone harvester.

56. The bone harvesting guide of inventive concept 54 or 55, wherein the first guiding pin extends longer by a distance than the second guiding pin at a distal end of the bone harvesting guide.

57. The bone harvesting guide of inventive concept 56, wherein the distance is about half a length of a bone core to be harvested by the bone harvester.

58. The bone harvesting guide of inventive concept 56, wherein the distance is from about 3 to about 9 mm.

59. The bone harvesting guide of inventive concept 54 or 55, wherein a distance between any two of the center of the first guiding pin, the center of the second guiding pin, and the center of the tube is from about 4 to about 9 mm.

60. The bone harvesting guide of inventive concept 54 or 55, wherein the first guiding pin has a length that is about 1 mm to about 12 mm greater than a length of the second guiding pin.

61. The bone harvesting guide of inventive concept 55, wherein a distal end of first guiding pin comprises a diameter, which is not longer than a diameter of a bone core to be harvested by the bone harvester.

62. The bone harvesting guide of inventive concept 55, wherein a distal end of second guiding pin comprises a diameter, which is not longer than a diameter of a bone core to be harvested by the bone harvester.

63. The bone harvesting guide of inventive concept 62, wherein the diameter of the bone core is from about 2.5 to about 10 mm.

64. The bone harvesting guide of inventive concept 54 or 55, wherein the first guiding pin and the second guiding pin independently have a diameter from about 0.25 mm to about 5 mm.

65. The bone harvesting guide of inventive concept 54 or 55, wherein the perpendicular distance between the center of the first guiding pin and the center of the second guiding pin is from about 3 mm to about 9 mm.

66. The bone harvesting guide of inventive concept 54 or 55, wherein the first guiding pin and the second guiding pin are attached to an end of the tube.

67. The bone harvesting guide of inventive concept 54 or 55, wherein the first guiding pin and the second guiding pin are attached to an outer wall of the tube.

68. The bone harvesting guide of inventive concept 54 or 55, wherein the tube has a length from about 50 mm to about 100 mm.

69. The bone harvesting guide of any one of inventive concepts 55-68, wherein the bone harvester comprises a shaft having a lumen, a distal end with a blade having a distal cutting edge, and a proximal end; and wherein the bone harvesting guide can be moved along the shaft of the bone harvester such that the first guiding pin can be positioned at selected locations relative to a distal end of the bone harvester.

70. The bone harvesting guide of inventive concept 69, wherein, while being placed within the tube at an operating position, the cutting edge of the bone harvester is at the same cross-section of a tip of the second guiding pin.

71. The bone harvesting guide of inventive concept 70, wherein, while being placed, a distance between the center of the first guiding pin or the center of the second guiding pin to a side wall of the distal end of the shaft is from about 3 to about 8 mm.

72. A kit for harvesting a bone core, comprising:
a bone harvester of any one of inventive concepts 1-53; and
a pushing tool, wherein the pushing tool comprises a pushing rod having a shape matching the lumen defined by the shaft.

73. The kit of inventive concept 72, wherein the pushing rod has a width matching a dimension the lumen defined by the shaft.

74. The kit of inventive concept 72, wherein the pushing rod has an outer diameter, which is smaller than the diameter of the lumen, such that the pushing rod can slide within the lumen.

75. The kit of inventive concept 72, wherein the pushing tool further comprises a blind tube having a closed end and an open end, wherein the blind tube defines an inner space having a same cross-sectional area along a longitudinal axis from the closed end to the open end; and wherein the pushing rod is positioned inside the inner space with an end thereof mounted at the closed end of the blind tube.

76. The kit of inventive concept 75, wherein the cross-sectional area of the inner space of the blind tube is substantially the same as a cross-sectional area of the distal end of the shaft, whereby the inner space of the blind tube is configured to accommodate the distal end of the shaft.

77. The kit of inventive concept 76, wherein the shaft further comprises a window, and the window is provided through a side wall thereof and is configured to expose a portion of the lumen; and further wherein the pushing rod further comprises a free end, which is not mounted at the blind tube, and the pushing rod is of a length that, when the blind tube is assembled with the shaft, the free end of the pushing rod is visible from the window of the shaft.

78. The kit of inventive concept 75, wherein when the blind tube is assembled with the shaft, the distal end of shaft is against an inner surface of the closed end of the blind tube.

79. The kit of inventive concept 72, wherein the pushing tool further comprises a head where the pushing rod is mounted.

80. The kit of inventive concept 79, wherein the shaft comprises a first portion and a second portion,
wherein the first portion is positioned proximal to the distal end thereof and comprises a smooth inner surface,
wherein the second portion is positioned proximal to the proximal end thereof and comprises a threaded inner surface, and the threaded inner surface comprises a first thread; and
wherein the pushing rod comprises a threaded portion having a diameter and a threaded outer surface suitable for screwing into the second portion.

81. The kit of inventive concept 80, wherein the threaded portion is positioned proximal to the proximal end of the pushing rod.

82. The kit of inventive concept 79, wherein the pushing tool further comprises a leading rod that is not attached to the pushing rod and has a shape matching the lumen defined by the shaft.

83. The kit of inventive concept 82, wherein the leading rod comprises a diameter that is no larger than the diameter of the lumen of the distal end.

84. The kit of any one of inventive concepts 72-84, further comprising a bone harvesting guide of any one of inventive concepts 54-71.

85. A kit for harvesting a bone core, comprising:
a bone harvesting guide according to any one of inventive concepts 54-71;
a bone harvester comprising a shaft having a lumen, a distal end with a blade having a distal cutting edge, and a proximal end;
wherein the tube of the bone harvesting guide is configured to accommodate the bone harvester.

86. The kit of inventive concept 85, wherein the first guiding pin extends longer by a distance than the second guiding pin at a distal end of the bone harvesting guide.

87. The kit of inventive concept 86, wherein the distance is not longer than a length of a bone core to be harvested by the bone harvester.

88. The kit of inventive concept 86, wherein the distance is from about 2 to about 12 mm.

89. The kit of inventive concept 85, wherein a distance between any two of the center of the first guiding pin, the center of the second guiding pin, and the center of the tube is from about 4 to about 9 mm.

90. The kit of inventive concept 85, wherein a distal end of first guiding pin comprises a diameter, which is not longer than a diameter of a bone core to be harvested by the bone harvester.

91. The kit of inventive concept 85, wherein a distal end of second guiding pin comprises a diameter, which is not longer than a diameter of a bone core to be harvested by the bone harvester.

92. The kit of inventive concept 90 or 91, wherein the diameter of the bone core is from about 1 to about 12 mm.

93. The kit of inventive concept 85, wherein the bone harvesting guide can be moved along the shaft of the bone harvester such that the first guiding pin can be positioned at selected locations relative to a distal end of the bone harvester.

94. The kit of inventive concept 85, wherein, while being assembled within the tube at an operating position, the cutting edge of the bone harvester is at the same cross-section of a tip of the first guiding pin.

95. The kit of inventive concept 85, wherein, while being assembled within the tube at an operating position, a distance between the center of the first guiding pin or the center of the second guiding pin to a side wall of the distal end of the shaft is from about 3 to about 7 mm.

96. A method for processing a bone, comprising:
inserting a bone harvester of any one of inventive concepts 1-53 into a bone whereby at least a portion of the engagement element is within the bone and the bone core is within the lumen;
rotating the bone harvester to rotate the bone core relative to the bone to shear the base of the bone core from the bone; and
withdrawing the bone harvester thereby obtaining a harvested bone core.

97. The method of inventive concept 96, wherein the portion of the engagement element comprises at least about 20% of the engagement element by its length.

98. The method of inventive concept 97, wherein, while rotating the bone harvester, the entire engagement element is within the bone.

99. The method of any one of inventive concepts 96-98, wherein inserting the bone harvester comprises inserting the cutting edge thereof into cartilage tissue or subchondral tissue, or a combination thereof of the bone.

100. The method of inventive concept 99, wherein inserting the bone harvester comprises contacting the cutting edge of the bone harvester with a surface of the cartilage tissue.

101. The method of any one of inventive concepts 96-100, wherein inserting the bone harvester comprises applying a compressive force to the proximal end of the shaft.

102. The method of inventive concept 101, wherein applying comprises hitting the proximal end of the shaft with a hammer or a mallet.

103. The method of any one of inventive concepts 96-102, wherein the bone harvester comprises a step adjacent to the blade and wherein the inserting is performed until the step contacts a surface of the bone.

104. The method of any one of inventive concepts 96-103, wherein withdrawing the bone harvester comprises removing the bone harvester out of the bone and leaving a hole at the bone.

105. The method of inventive concept 104, wherein the hole has a shape corresponding to the harvested bone core.

106. The method of any one of inventive concepts 96-105, wherein the harvested bone core has a diameter from about 1 to about 12 mm.

107. The method of inventive concept 106, wherein the diameter of the bone core is about 1 to 5 mm.

108. The method of any one of inventive concepts 96-107, wherein the harvested bone core has a length from about 2 to about 12 mm.

109. The method of inventive concept 108, wherein the harvested bone core has a length of about 4 to 10 mm.

110. The method of any one of inventive concepts 96-109, further comprising removing the harvested bone core out of the bone harvester.

111. The method of any one of inventive concepts 96-110, wherein the harvested bone core is a first harvested bone core, and the method further comprises harvesting a second bone core.

112. The method of inventive concept 111, wherein, after the first harvested bone core is obtained and a first hole is left on the bone, the method further comprises
inserting a bone harvesting guide of any one of inventive concepts 54-71 within the bone whereby the first guiding pin of the bone harvesting guide is inserted into the first hole;
assembling the bone harvester with the bone harvesting guide;
inserting the bone harvester into the bone whereby a portion of the open slot of the bone harvester is within the bone, and the first guiding pin of the bone harvesting guide is maintained inside the first hole;
rotating the bone harvester while the portion of the open slot maintains within the bone; and
withdrawing the bone harvester thereby obtaining a second harvested bone core and leaving a second hole on the bone.

113. The method of inventive concept 112, wherein, after the second harvested bone core is obtained and the second hole is left on the bone, the method further comprises
inserting the bone harvesting guide into the bone whereby the first guiding pin of the bone harvesting guide is inserted into one of the first hole or the second hole;
assembling the bone harvester with the bone harvesting guide;
inserting the bone harvester into the bone whereby a portion of the open slot of the bone harvester is within the bone;
rotating the bone harvester while the portion of the open slot maintains within the bone; and
withdrawing the bone harvester thereby obtaining a third harvested bone core and leaving a third hole on the bone.

114. The method of inventive concept 113, wherein the first hole, the second hole, and the third hole are substantially equidistant from each other.

115. The method of inventive concept 114, wherein the first hole, the second hole, and the third hole are substantially equidistant from each other at a distance from about 4 to about 9 mm.

116. The method of any one of inventive concepts 96-115, wherein rotating the bone harvester comprises rotating the bone harvester by about 5 to about 360 degrees relative to the bone.

117. The method of any one of inventive concepts 96-115, wherein rotating the bone harvester comprises rotating the bone harvester by at least about 45 degrees relative to the bone.

118. The method of any one of inventive concept 96-117, wherein the bone harvester further comprises a handle module connected to the proximal end of the shaft; and further wherein rotating the bone harvester is achieved by operating the handle module.

119. The method of any one of inventive concepts 96-118, wherein rotating the bone harvester comprises rotating the bone harvester clock-wisely.

120. The method of any one of inventive concepts 96-119, wherein obtaining a harvested bone core comprises removing the harvested bone core from the bone harvester.

121. The method of inventive concept 120, wherein the removing comprises inserting a pushing tool into the lumen at the proximal end to push the harvested bone core out of the distal end.

122. The method of inventive concept 121, wherein inserting the pushing tool comprises screwing a pushing tool into the lumen at the proximal end,
wherein the lumen comprises a first lumen portion and a second lumen portion, wherein the first lumen portion comprises a smooth inner surface and the second lumen portion comprises a threaded inner surface; and
wherein the pushing tool has a pushing rod, wherein the pushing rod comprises a threaded portion having a diameter and a threaded outer surface suitable for screwing into the second lumen portion.

123. The method of inventive concept 120, wherein the bone harvester further comprises a window proximal to the blade, wherein the window comprises an opening on a side portion of the shaft, wherein the distal portion of the opening connects to the lumen of the blade, and wherein the removing comprises inserting a pushing tool into the lumen of the blade to direct the harvested bone core out of the window.

124. The method of any one of inventive concepts 96-123, wherein the bone is of a patient having cartilage defects.

125. The method of any one of inventive concepts 96-124, wherein the bone has a partial thickness defect or full thickness defect.

126. The method of any one of inventive concepts 96-125, wherein the method is performed through an arthroscopy procedure.

127. A bone core comprising a distorted cylindrical body, wherein the distorted cylindrical body has a non-circular cross section perpendicular to the cylindrical axis corresponding to an accretion to or removal from the corresponding circular form.

128. The bone core of inventive concept 127, wherein the distorted cylindrical body comprises a ridge portion extending from a side of the distorted cylindrical body, a channel protruding into a side of the distorted cylindrical body, or a flat portion along a side of the distorted cylindrical body, or a combination thereof.

129. The bone core of inventive concept 127 or 128, wherein the cylindrical body has a diameter of about 1 mm to about 12 mm.

130. The bone core of inventive concept 129, wherein the cylindrical body has a diameter from about 1 mm to about 5 mm.

131. The bone core of any one of inventive concepts 127-130, wherein the cylindrical body has a length of about 2 mm to about 12 mm.

132. The bone core of inventive concept 131, wherein the cylindrical body has a length of about 4 to 10 mm.

133. The bone core of any one of inventive concepts 127-132, wherein the bone core comprises cartilage tissue, subchondral tissue, or a combination thereof.

134. The bone core of any one of inventive concepts 127-133, wherein the bone core comprises a layer of cartilage tissue and a layer of subchondral tissue.

135. The bone core of any one of inventive concepts 127-134, wherein the bone core comprises damaged cartilage tissue or healthy cartilage tissue.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understand that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated. The use of the term “about” herein refers to expected uncertainties in the associated values as would be understood in the particular context by a person of ordinary skill in the art.

What is claimed is:

1. A bone harvester for harvesting a bone core, the bone harvester comprising a shaft having a lumen, a distal end with a blade having a distal cutting edge, a proximal end, and an engagement element that locks the relative rotation of the bone harvester and the bone core.

2. The bone harvester of claim 1 wherein the engagement element comprises an open slot, a notch, a flat face, an internal slot substantially parallel to a longitudinal axis of the shaft, a collar, a plurality thereof, or a combination thereof.

3. The bone harvester of claim 2 wherein the engagement element comprises the open slot, and the open slot has a side opening through the blade that extends from the distal cutting edge toward the proximal end of the shaft.

4. The bone harvester of claim 2 wherein a length of the open slot is at least 20% of a length of the bone core.

5. The bone harvester of claim 2 wherein a width of the open slot is from 5% to 10% of the circumference of the bone core.

6. The bone harvester of claim 2, wherein the open slot has a width from about 0.3 mm to about 7 mm.

7. The bone harvester of claim 2 wherein the engagement element comprises the notch, and the notch comprises a protrusion into the lumen of the shaft.

8. The bone harvester of claim 1 wherein the length of the bone core is about 4 to 10 mm.

9. The bone harvester of claim 1 wherein the bone core has a diameter from about 2.5 to about 8 mm.

10. The bone harvester of claim 1 wherein the blade has a circumferential shape and has a longitudinal through-bore connecting to the lumen of the shaft.

11. The bone harvester of claim 1 wherein the blade has a tapered outer diameter along a direction of an axis of the shaft toward the distal end.

12. The bone harvester of claim 1 wherein the blade has an outer diameter that tapers by a amount from about 3 mm to about 5 mm, such that the proximal diameter is larger than the distal diameter.

13. The bone harvester of claim 1, wherein the blade has a tapered wall thickness.

14. The bone harvester of claim 1 wherein the shaft comprises a step adjacent to the blade, wherein the step is formed by a change in outer diameter of the shaft.

15. The bone harvester of claim 14 wherein the step has a position to form a bone core having a length from about 2 mm to about 12 mm.

16. The bone harvester of claim 1 wherein the shaft further comprises a window proximal to the blade, wherein the window comprises an opening on a side portion of the shaft to expose a portion of the lumen.

17. The bone harvester of claim 1 further comprising a handle comprising a handle module, wherein the handle module is mechanically attached to the shaft and one or more rods, wherein the handle module comprises a longitudinal hole extending through the handle module and one or more lateral holes, wherein the shaft extends through the longitudinal hole and each of the one or more rods extends from a lateral hole approximately perpendicular to a longitudinal axis of the shaft.

18. A bone harvesting guide comprising:
a first guiding pin;
a second guiding pin; and
a tube configured to accommodate a bone harvester;
wherein the first guiding pin, the second guiding pin, and the tube are assembled whereby a longitudinal axis of the first guiding pin, a longitudinal axis of the second guiding pin, and a longitudinal axis of the tube are substantially parallel to each other; and
wherein, in a cross-section of the bone harvesting guide, a center of the first guiding pin, a center of the second guiding pin, and a center of the tube form vertexes of an equilateral triangle.

19. A kit for harvesting a bone core comprising:
a bone harvester according to claim 1; and
a pushing tool, wherein the pushing tool comprises a pushing rod having a shape matching the lumen defined by the shaft.

20. A method for processing a bone comprising:
inserting a bone harvester according to claim 1 into a bone whereby at least a portion of the engagement element is within the bone and the bone core is within the lumen;
rotating the bone harvester to rotate the bone core relative to the bone to shear the base of the bone core from the bone; and
withdrawing the bone harvester thereby obtaining a harvested bone core.

21. A bone core comprising a distorted cylindrical body, wherein the distorted cylindrical body has a non-circular cross section perpendicular to the cylindrical axis corresponding to an accretion to or removal from the corresponding circular form.
